(19) United States
(12) Patent Application Publication
(10) Pub. No.: US 2008/0280920 A1
(43) Pub. Date: Nov. 13, 2008

(54) Plasminogen Activator Inhibitor-1 Inhibitors

(76) Inventors: Latchezar S. Trifonov, Montreal (CA); Jean Vaugeois, Montreal (CA); Bhavna Gaikwad, New Haven, CT (US); Robert S. Greenfield, Trumbull, CT (US)

Correspondence Address: FROMMER LAWRENCE & HAUG, 745 FIFTH AVENUE- 10TH FL., NEW YORK, NY, 10151, US

(21) Appl. No.: 12/096,479

(22) PCT Filed: Dec. 7, 2006
(86) PCT No.: PCT/CA2006/001990

§ 371 Date: Jun. 6, 2008

Related U.S. Application Data
(60) Provisional application No. 60/742,882, filed on Dec. 7, 2005.

(51) Int. Cl. A61K 31/506; A61K 31/496; C07D 403/14; A61P 35/00; A61P 9/00; A61P 9/10; C07D 401/04

(52) U.S. Cl. 514/252.19; 544/360; 514/253.01; 544/295

(57) ABSTRACT

Inhibitors of plasminogen activator inhibitor-1 (PAI-I) are provided, which may also act as anti cancer agents, of formulae (I-V).

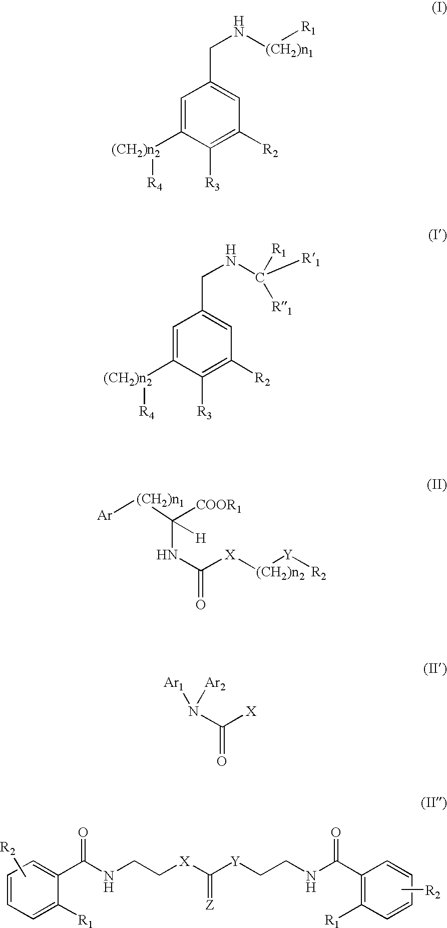

This application claims priority from U.S. provisional application 60/742,882, the content of which is incorporated herein by reference.

### FIELD OF THE INVENTION

The invention relates generally to pharmaceutical compounds. In particular, the invention relates to pharmaceutical compounds which act as inhibitors of plasminogen activator inhibitor-1 (PAI-1) as well as anti cancer agents.

### BACKGROUND

Plasminogen activator inhibitor-1 (PAI-1) is a single-chain glycoprotein (379-381 amino-acids, ˜45 kDa) which belongs to the serine protease inhibitor superfamily¹. In vivo, two serine proteases, tissue-plasminogen activator (tPA) and urokinase-plasminogen activator (uPA) convert plasminogen, an inactive zymogen, into the active enzyme plasmin, which digests fibrin clots by degrading insoluble fibrin molecules into smaller soluble fragments (fibrinolysis). PAI-1 inhibits both tPA and uPA making it a key regulator of the fibrinolytic system. PAI is produced in human endothelial cells, platelets, placenta, hepatocytes, vascular smooth muscle cells, mesangial cells, fibroblasts, monocytes/macrophages and plentifully by stroma cells from adipose tissue². Elevated PAI-1 activity reduces fibrinolytic potential, promotes fibrin deposition in the vasculature and contributes to the development of thrombotic and thromboembolic diseases including recurrent deep vein thrombosis, disseminated intravascular coagulation, unstable angina, myocardial infarction and coronary artery disease. In healthy individuals plasma PAI-1 levels are low but they may be elevated significantly in several disease states including venous thromboembolism, atherosclerosis, metabolic syndrome and type 2 diabetes³. Plasma PAI-1 is also elevated in post-menopausal women and has been proposed to contribute to the increased incidence of cardiovascular disease in this population⁴.

Aside from its conventional role in inhibiting tPA-mediated plasminogen activation and promoting the dissolution of fibrin clots in the circulation, PAI-1 appears to have significant effects on cell adhesion, detachment and migration of normal cells as well as invasion and metastasis of cancer cells². One likely mechanism for this is via direct PAI-1 binding of vitronectin (Vn)⁵, an extra-cellular matrix (ECM) protein which has a high-affinity PAI-binding motif at amino acids 12-30². In the absence of PAI, uPA bound to its receptor uPAR occupies this motif on vitronectin, an effect competitively inhibited by PAI-1. Binding of PAI-1 to vitronectin dissociates the uPA-Vn interaction causing enhanced activation of cell-bound plasminogen and local proteolysis of the ECM thereby altering cell adhesion and migration. Vitronectin can also bind integrin \acute{α}₅β₃, a molecule involved in cell adhesion⁶. PAI-1 can detach cells from the ECM and contribute to tissue remodeling by disrupting uPAR-Vn and integrin-Vn interactions^{7,8}. Thereby PAI-1 may play a role in cell adhesion or migration as well as cancer invasiveness via a mechanism independent of its anti-proteolytic activity⁹. Therefore, high levels of PAI-1 have been associated with poor patient prognosis in a variety of cancers including invasive node-negative breast cancer, prostate carcinoma, squamous-cell carcinoma, adenocarcinoma of the lung and colo-rectal cancers^{10,11}.

In animal studies, transgenic mice expressing high levels of PAI-1 develop spontaneous thrombosis whereas PAI-1 deficient mice are more resistant to venous or arterial thrombosis as well as atherogenesis¹². Inhibition of PAI-1 activity by monoclonal antibodies prevents thrombus formation in animal models without directly affecting blood coagulation or platelet function, indicating that inhibition of PAI-1 is a valuable potential strategy to prevent thrombosis. Pharmacological inhibition of PAI-1 activity prevents arterial and venous thrombosis in animal models and has been evaluated as a novel approach to anti-thrombotic drugs¹³. An antithrombotic agent based on PAI-1 inhibition may have a lower risk of bleeding than that of conventional antiplatelet and anticoagulant drugs.

In experimental animal models, PAI-1 has been shown to regulate tumor invasiveness and growth as well as angiogenesis^{1,14}. Pharmacological inhibition of PAI-1 has been demonstrated to prevent cancer invasion and vascularization. These results suggest that modulation of PAI-1 activity offers a beneficial therapy in treating a variety of thrombotic and fibrinolytic disorders¹⁵, tissue remodeling disorders involve the cardiovascular, renal and respiratory systems, as well as a variety of cancers involving extra-cellular matrix invasion and angiogenesis^{4,16,17}.

Increased levels of PAI-1 were observed in the metabolic syndrome and a significant correlation was found between plasma PAI-1 levels and body mass index, triglyceride levels, insulin levels/resistance and systolic blood pressure¹⁸. Adipose tissue contributes significantly to plasma PAI-1 levels and PAI-1 inhibits insulin signaling by competing with integrin \acute{α}₅β₃ for binding to vitronectin^{19,20}. Improvement in the symptoms of metabolic syndrome by diet, exercise or oral anti-diabetic drugs such as the thiazolidinediones improved lipid profile, enhanced fibrinolytic activity and reduced PAI-1 levels²⁰. These results indicate a potential therapeutic use of PAI-1 inhibitors for treating Type 2 diabetes and the metabolic syndrome.

PAI-1 has multiple functions including anti-proteolysis, binding to vitronectin and interference with cell migration and ECM binding²¹ have revealed its involvement in a variety of physiologic and pathophysiologic events such as wound healing, atherosclerosis, metabolic disturbances, tumor proliferation and angiogenesis, chronic stress, bone and blood vessel-wall remodeling, asthma, rheumatoid arthritis, sepsis, glomerular nephritis, lung fibrosis, polycystic ovary disease etc². Pharmacologic inhibition of PAI-1 therefore provides a promising avenue of drug development for a wide variety of thrombogenic and fibrotic disorders^{13,22,23,24,25,26}.

### SUMMARY OF THE INVENTION

The present invention therefore relates to compounds that inhibit PAI-1. The compounds of the invention inhibit the interaction of PAI-1 with uPA, thereby enhancing and prolonging the action of uPA. It is believed that these compounds show low inhibitory activities towards PAI-1's interaction with vitronectin.

The invention provides according to a first aspect, for a compound of formula I or I′:

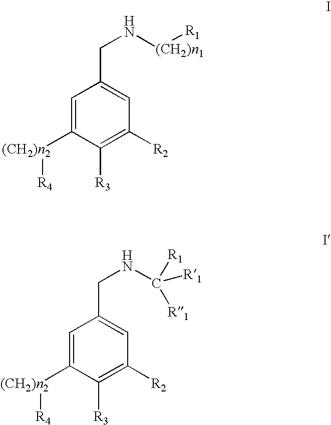

In the above, R₁, R′₁ and R″₁ are each independently selected from H; linear, branched, saturated, unsaturated, cyclic, bicyclic, fused, substituted or unsubstituted alkyl; and an aryl, arylalkyl or heterocyclic group which is optionally substituted. R₂ and R₃ are each independently selected from H; OH; SH; alkoxy; alkylthio; aryloxy and arylthio. R₄ is a linear, branched, saturated, unsaturated, cyclic, bicyclic, fused, substituted, or unsubstituted alkyl; an aryl or arylalkyl group optionally containing at least one hetero atom and/or being aromatic and/or being substituted or unsubstituted; or a 4-substituted or unsubstituted piperazin-1-yl group. The values for n₁ and n₂ are each independently selected from 0 to 6. The stereochemistry of the carbon atom in I′ bearing substituents R₁, R′₁ and R″₁ is S or R. Compounds of formulae I or I′ also comprise any pharmaceutically acceptable salt thereof.

According to the above aspect, the compound can be Ia or I′a:

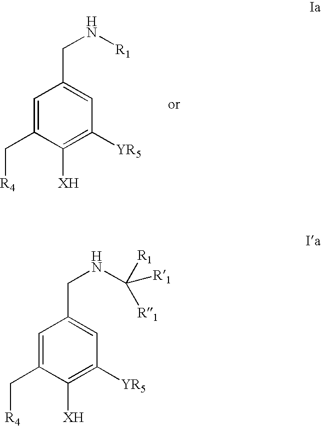

In the above, R₁, R′₁, R″₁, and R₄ are as defined above. R₅ is a linear, branched, saturated or unsaturated alkyl; or an aryl or arylalkyl which is optionally substituted. X and Y are each independently selected from O and S. The stereo-chemistry of the carbon atom bearing substituents R₁, R′₁, and R″₁ is S or R. Compounds of Ia and I′a also comprise any pharmaceutically acceptable salt thereof.

The compound can also be Ib or I′b:

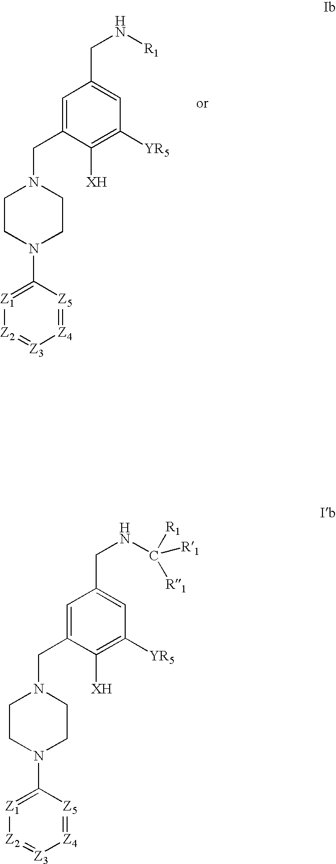

In the above, R₁, R′₁, R″₁, R₅, X and Y are as defined above. Z₁ to Z₅ are each independently selected from C and N, and the stereochemistry of the carbon atom bearing substituents R₁, R′₁ and R″, is S or R. Compounds Ib and I′b also comprise any pharmaceutically acceptable salt thereof.

Still according to this aspect, the compound can be A, B, C or D

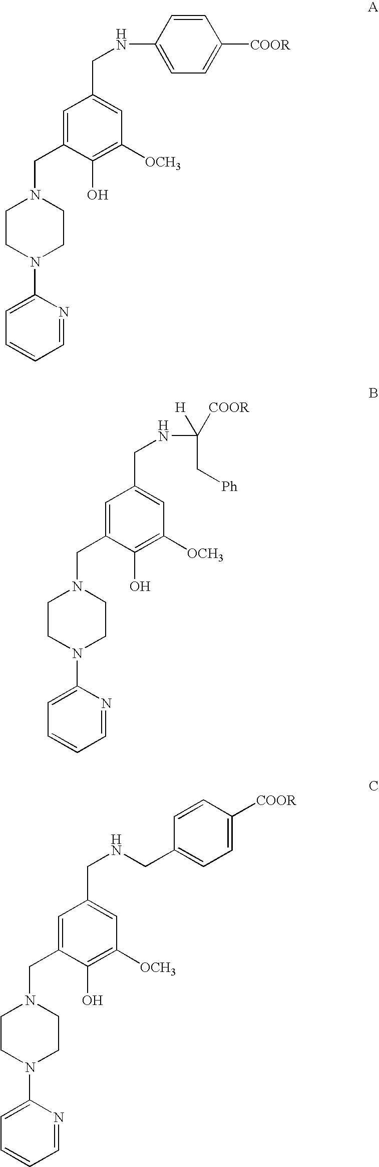

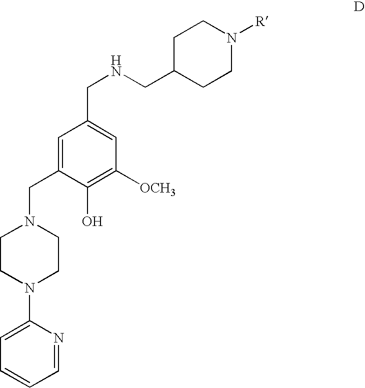

wherein R and R′ are each independently selected from H and a linear, branched, saturated or unsaturated alkyl, and optionally R′is Boc or C(═NH)NH₂; and the stereochemistry of the asymmetric carbon is S or R, or a pharmaceutically acceptable salt thereof.

Optionally, substituent R of compounds according to this first aspect may be H, Et or t-Bu, and substituent R′ may be H, Boc or C(═NH)NH₂.

The compound can also be selected from

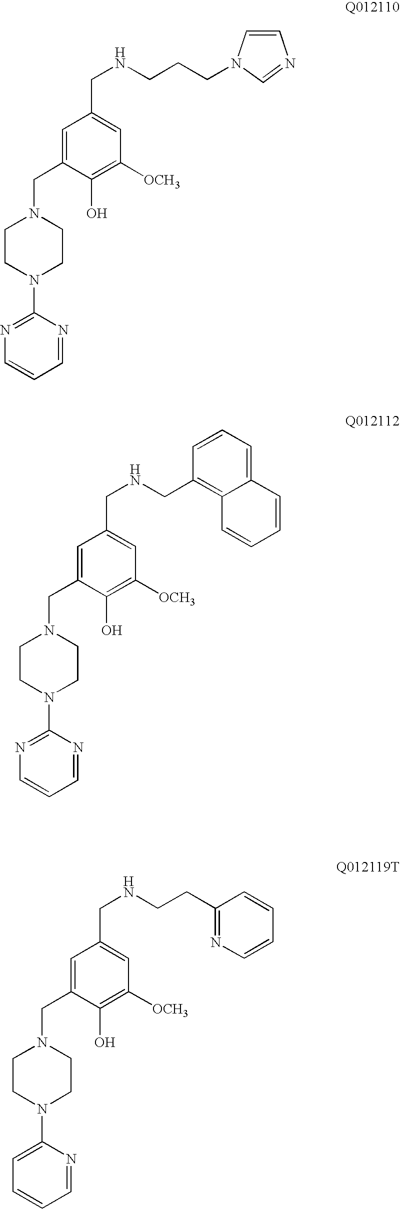

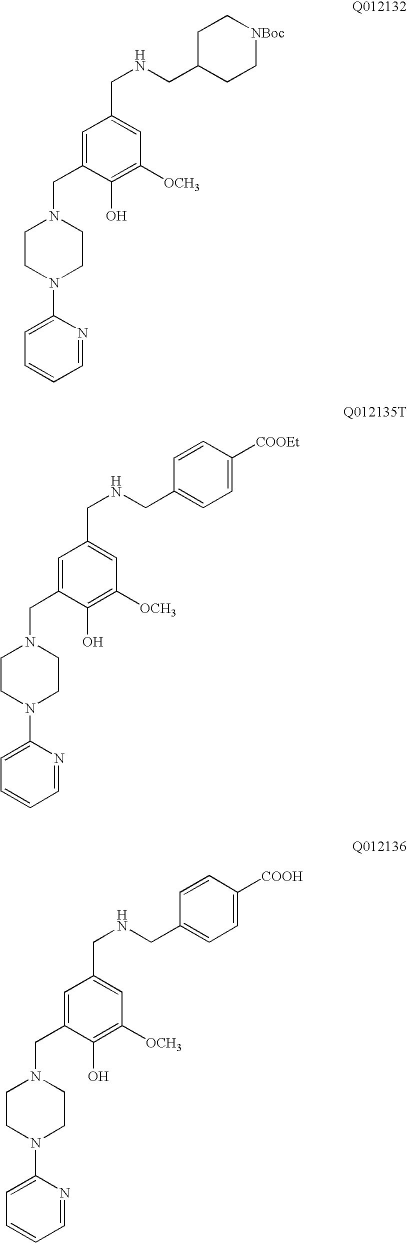

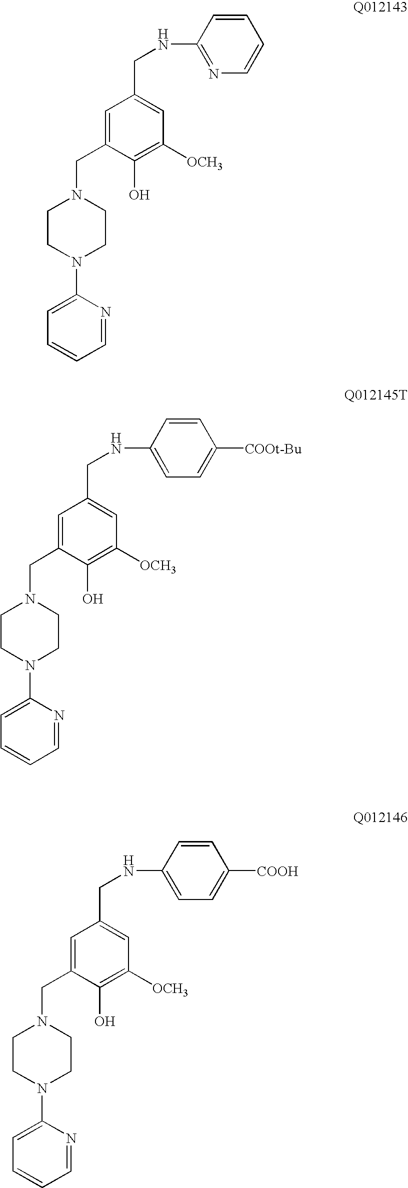

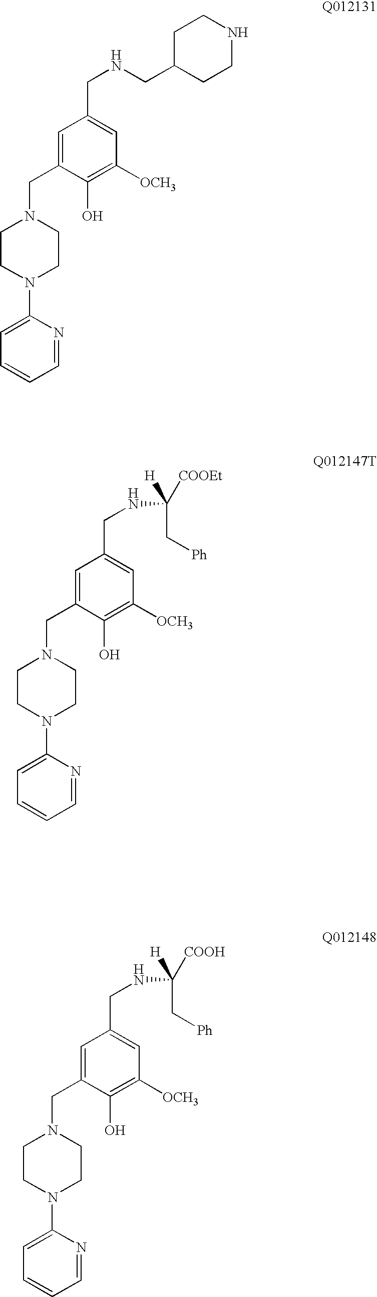

The invention provides according to a second aspect, for a compound of formula II or II′:

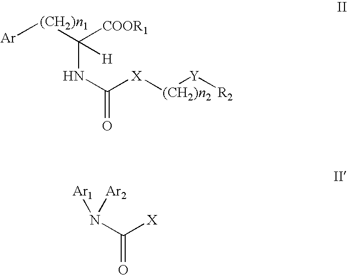

In the above, Ar, Ar₁ and Ar₂ are each independently selected from a cyclic, bicyclic, fused, substituted or unsubstituted alkyl, aryl or arylalkyl group both or either of which may optionally contain at least one heteroatom; a substituted or unsubstituted phenyl or aryl optionally containing at least one heteroatom; thianaphthenyl or indolyl. Optionally Ar₁ and Ar₂ together form a substituted, unsubstituted, saturated, unsaturated or an aromatic carbo cycle which optionally contains at least one heteroatom. Optionally Ar, Ar₁ and Ar₂ contain a guanidyl or (arylsulfonyl)guanidyl group. R₁ is H, alkyl, aryl or arylalkyl. R₂ is H; a cyclic, bicyclic, fused, substituted or unsubstituted alkyl, aryl or arylalkyl group optionally containing at least one heteroatom; or substituted or unsubstituted phenyl, pyridyl, 2-oxo-H-chromenyl. X is H; CH₂; or CHR₃R₄, R₃ and R₄ being each independently selected from H, acid or ester group, linear or branched alkyl group optionally containing a hetero-atom, or together R₃ and R₄ form a substituted, unsubstituted, saturated, unsaturated or aromatic carbo cycle which optionally contains at least one heteroatom. Y is CH₂, O, S, an alkyl group optionally containing at least one heteroatom, guanidyl, or (arylsulfonyl) guanidyl. Values for n₁ and n₂ are each independently selected from 0 to 6. Compounds II and II′ also comprise any or a pharmaceutically acceptable salt of the above.

According to this second aspect, the invention also provides for a compound of formula II″ or II′″:

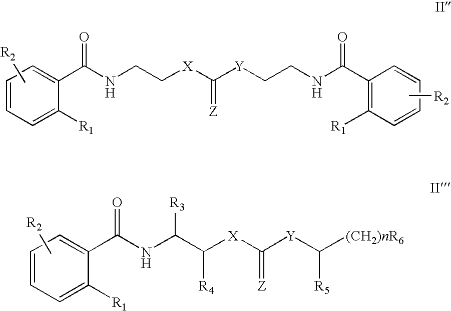

In the above, R₁ and R₂ are each independently selected from H, OH, SH, alkoxy, alkylthio, aryloxy and arylthio. R₃ and R₄ are each independently selected from H, a linear, branched, saturated, unsaturated, cyclic, bicyclic, fused, substituted, or unsubstituted alkyl and an aryl or arylalkyl group optionally containing at least one hetero atom and/or being aromatic and/or being substituted or unsubstituted. R₅ and R₆ are each independently selected from H, OH, SH, alkoxy, alkylthio, aryloxy, arylthio, NH₂, COOH, COOalkyl, COOarylalkyl and COOaryl, optionally R₆ contains a NH—C(NHR₇)═NH group, wherein R₇ is H or SO₂Ar. X, Y and Z are each independently selected from O, S and NH. The value of n is selected from 0 to 6. Compounds of II″ and II′″ also comprise a stereoisomer thereof, or a pharmaceutically acceptable salt thereof.

Optionally, a compound according to this second aspect can be selected from IIa or IIb:

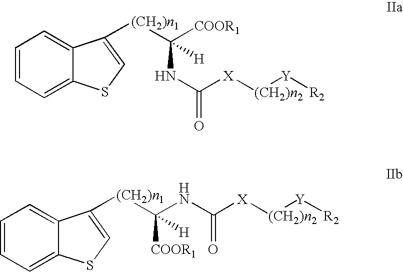

wherein: R₁ is H, alkyl, aryl or arylalkyl; R₂ is H; a cyclic, bicyclic, fused, substituted or unsubstituted alkyl, aryl or arylalkyl group optionally containing at least one heteroatom; or substituted or unsubstituted phenyl, pyridyl, 2-oxo-H-chromenyl; X is H; CH₂; or CHR₃R₄, R₃ and R₄ being each independently selected from H, acid or ester group, linear or branched alkyl group optionally containing a hetero-atom, or together R₃ and R₄ form a substituted, unsubstituted, saturated, unsaturated or aromatic carbo cycle which optionally contains at least one heteroatom; Y is CH₂, O, S, an alkyl group optionally containing at least one heteroatom, guanidyl, or (arylsulfonyl)guanidyl; and n₁ and n₂ are each independently selected from 0 to 6, or a pharmaceutically acceptable salt thereof.

The compound according to this second aspect can also be selected from IIc to IIl:

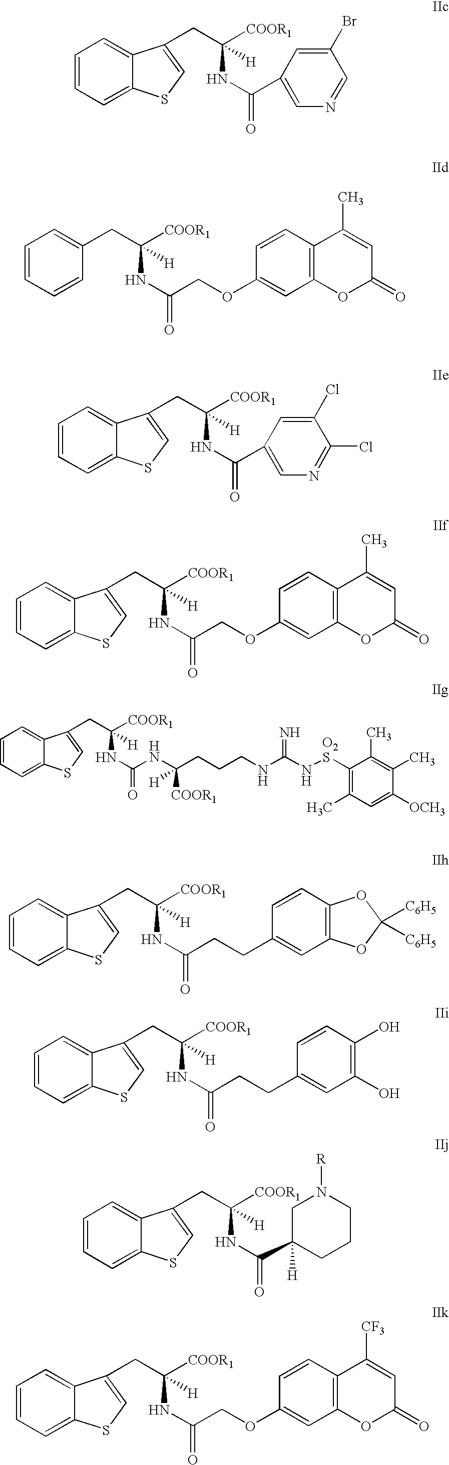

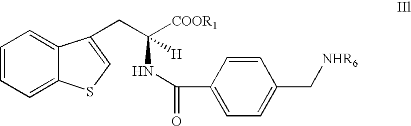

wherein R and R₆ are each independently selected from H, Boc and C(═NH)NH₂, or a stereoisomer or diastereoisomer thereof, or a pharmaceutically acceptable salt thereof.

Optionally, substituent R₁ of compounds according to this second aspect can be selected from H and a linear, branched, saturated or unsaturated alkyl, arylalkyl or aryl, and R₆ is H, Boc or C(═NH)NH₂, and substituent R₆ can be selected from H, Boc and C(═NH)NH₂.

The compound according to the second aspect can also be selected from

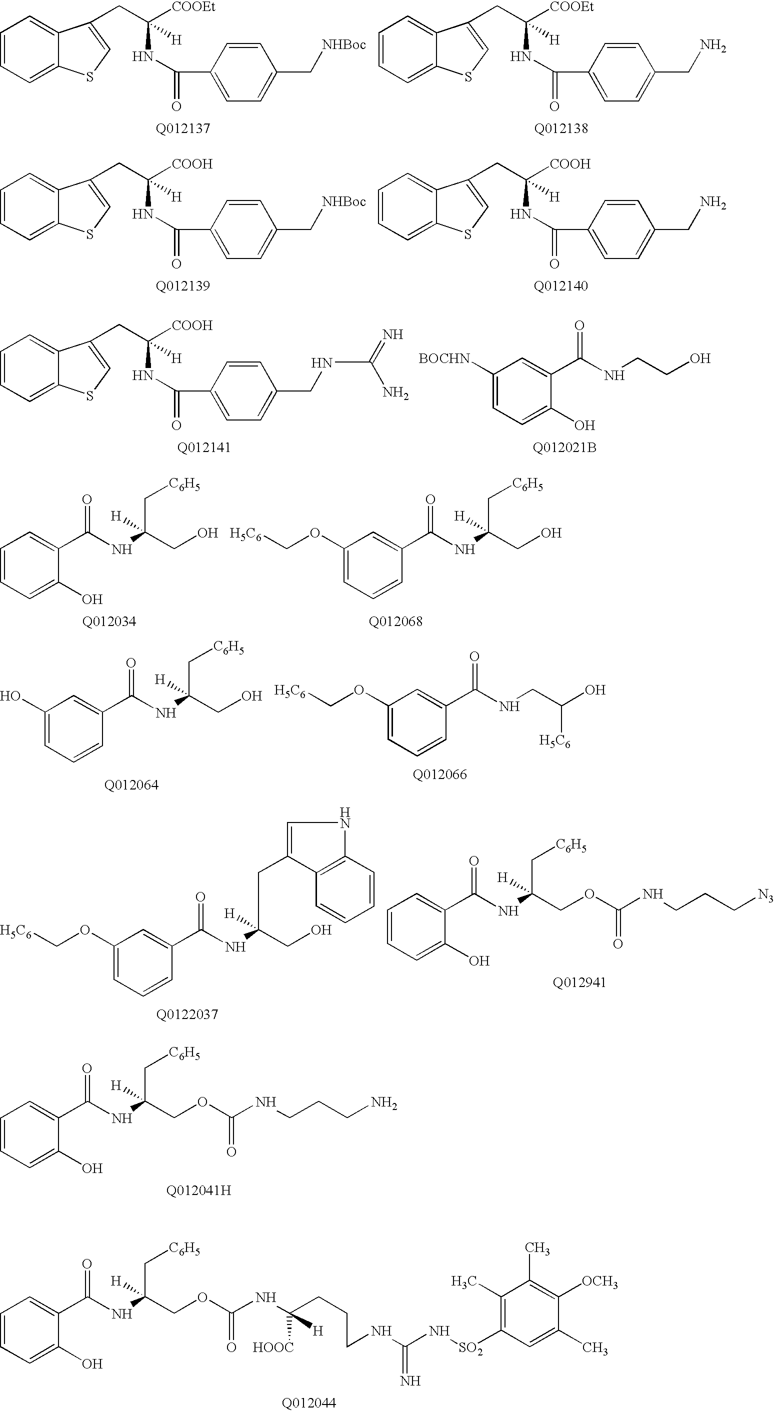

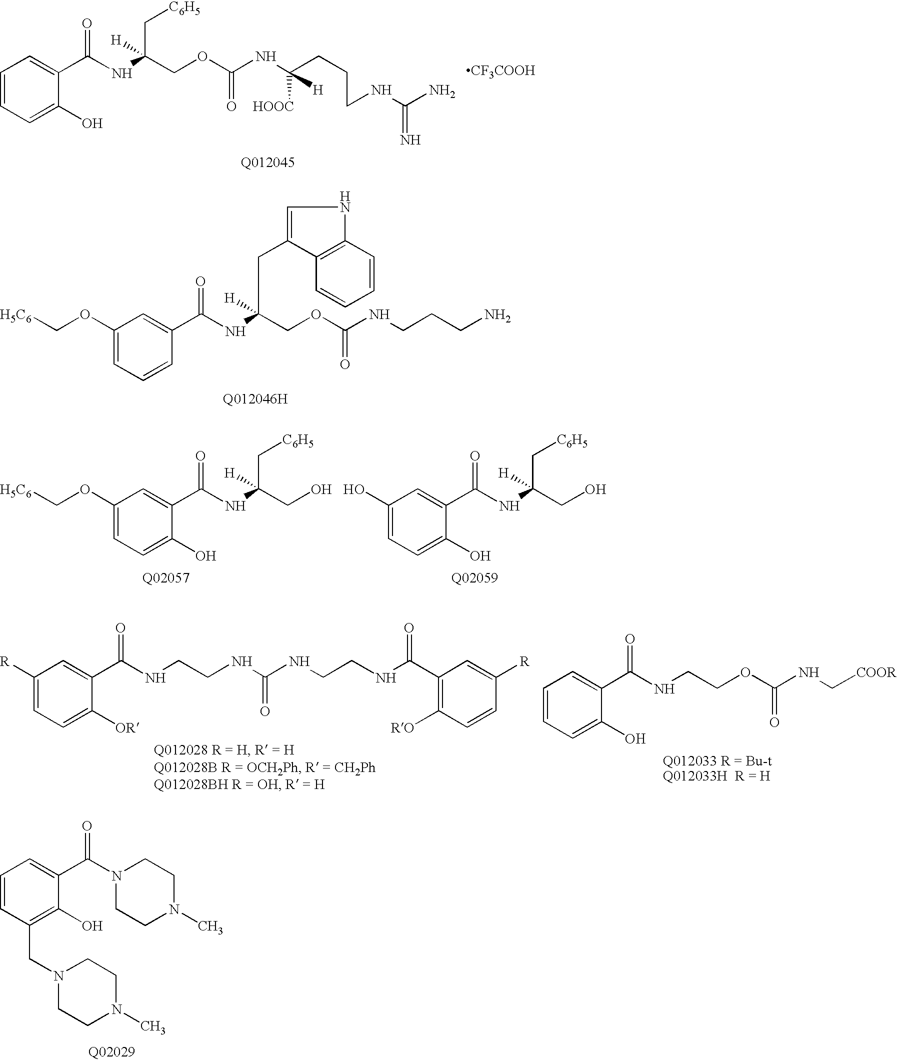

The invention provides according to a third aspect, for a compound of formula III:

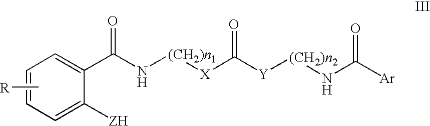

wherein: R is H, alkylamino, acylamino, or alkoxycarbonylamino; Ar is a substituted or unsubstituted phenyl or aryl optionally containing at least one hetero-atom; or a cyclic, bicyclic, fused, substituted or unsubstituted alkyl, aryl or aryl-alkyl group optionally containing at least one heteroatom; X and Y are each independently selected from O, S and NH; Z is O or S; and n₁ and n₂ are each independently selected from 0 to 6, or a pharmaceutically acceptable salt thereof.

According to this third aspect, the invention also provides for a compound of formula IIIa:

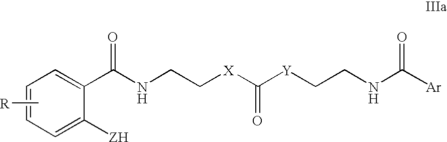

wherein: R is H, amino, alkylamino, acylamino, or alkoxycarbonylamino; Ar is a substituted or unsubstituted phenyl or aryl optionally containing at least one heteroatom; or a cyclic, bicyclic, fused, substituted or unsubstituted alkyl, aryl or arylalkyl group optionally containing at least one heteroatom; X and Y are each independently selected from O, S and NH; and Z is O or S, or a pharmaceutically acceptable salt thereof.

Optionally, a compound according to this third aspect can be Q012052

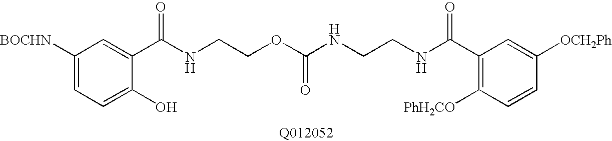

or a pharmaceutically acceptable salt thereof.

The invention provides according to a fourth aspect, for a compound of formula IV:

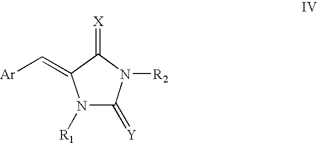

wherein: Ar is a substituted or unsubstituted phenyl or aryl optionally containing at least one heteroatom; or a cyclic, bicyclic, fused, substituted or unsubstituted alkyl, aryl or arylalkyl group optionally containing at least one heteroatom; R₁ and R₂ are each independently selected from H, a substituted or unsubstituted alkyl, aryl or arylalkyl, optionally containing at least one hetero-atom; X is O or S; and Y is O, S or NH, or a pharmaceutically acceptable salt thereof.

According to this fourth aspect, the invention also provides for a compound of general formula IVa or IVb:

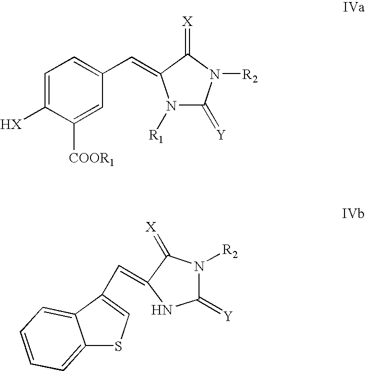

wherein: R₁ and R₂ are each independently selected from H, alkyl, aryl and arylalkyl; X is O or S; and Y is O, S or NH, or a pharmaceutically acceptable salt thereof.

Optionally, a compound according to this fourth aspect can be selected from

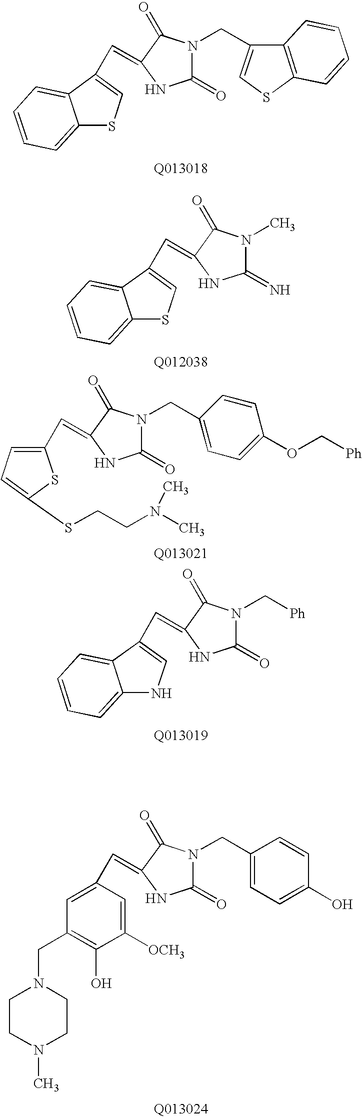

The invention provides according to a fifth aspect, for a compound of formula V:

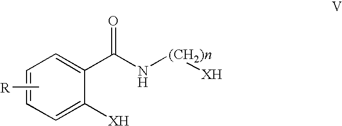

wherein: R is H, amino, alkylamino, acylamino or alkoxycarbonylamino; X is O or S; and n is selected from 0 to 6, or a pharmaceutically acceptable salt thereof.

According to this fifth aspect, the invention also provides for a compound of formula Va:

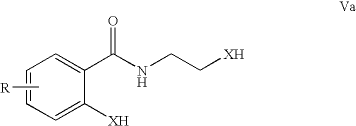

wherein: R is H, alkylamino or acylamino; and X is O or S, or a pharmaceutically acceptable salt thereof.

Optionally, a compound according to this fifth aspect is

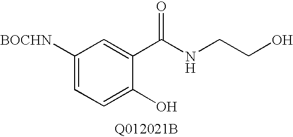

or a pharmaceutically acceptable salt thereof.

The invention also relates to pharmaceutically acceptable salts of the above compounds, as well as pharmaceutical compositions and preparations which comprise one or more of these compounds.

In all of the compounds described herein, “alkyl” preferably contains between 1 and 8 carbon atoms and most preferably between 1 and 6 carbon atoms. “Aryl” preferably comprises phenyl unless otherwise specified. Preferred substituents of substituted groups comprise the substituents in the specific compounds described or shown herein, wherein said substituents may be substituted on other groups in the compounds of this invention.

Furthermore, the invention relates to stereoisomers or diastereoisomers as well as any enol forms of the compounds according to the invention.

Salt forms of the compounds include but are not limited to sodium, potassium, calcium, magnesium salts as well as hydrochlorides, hydrobromides and sulfates. Salt forms of the compounds may also be salts of organic acids including acetic, fumaric, maleic, citric, tartaric salts, or the like. Other useful salt forms of these compounds may comprise pharmaceutically acceptable inorganic and organic bases including hydroxides, carbonates or bicarbonates of the therapeutically acceptable alkali metals or alkaline earth metals, such as sodium potassium, magnesium, calcium and the like. Acceptable organic bases include amines, such as benzylamine, mono-, di- and trialkylamines, preferably those having alkyl groups of form 1 to 6 carbon atoms, more preferably 1 to 3 carbon atoms, such as methylamine, dimethylamine, trimethylamine, ethylamine, diethylamine, triethylamine, mono-, di-, and triethanolamine. Also useful are alkylene diamines containing up to 6 carbon atoms, such as hexamethylenediamine; cyclic saturated or unsaturated bases containing up to 6 carbon atoms, including pyrrolidine, piperidine, morpholine, piperazine and their N-alkyl and N-hydroxyalkyl derivatives, such as N-methyl-morpholine and N-(2-hydroxy-ethyl)-piperidine, or pyridine. Quaternary salts may also be formed, such as tetraalkyl forms, such as tetramethyl forms, alkyl-alkanol forms, such as methyl-triethanol or trimethyl-monoethanol forms, and cyclic ammonium salt forms, such as N-methylpyridinium, N-methyl-N-(2-hydroxyethyl)-morpholinium, N,N-dimethylmorpholinium, N-methyl-N-(2-hydroxyethyl)-morpholinium, or N,N-dimethyl-piperidinium salt forms.

The compounds of the present invention are useful in the treatment, inhibition, prevention or prophylaxis in a mammal, preferably in a human, of diseases or conditions which involve the production and/or action of PAI-1. The compounds are useful in the treatment or prevention of one or more of the following: noninsulin dependent diabetes mellitus and cardiovascular disease caused by such conditions; prevention of thrombotic events associated with coronary artery and cerebrovascular disease; inhibiting the disease process involving the thrombotic and prothrombotic states which include atherosclerotic plaques, venous and arterial thrombosis, myocardial ischemia, atrial fibrillation, deep vein thrombosis, blood clotting disorders, pulmonary fibrosis, cerebral thrombosis, thromboembolic complications of surgery (eg. joint replacement), and peripheral arterial occlusion; stroke associated with or resulting from atrial fibrillation; diseases associated with extracellular matrix accumulation, including, but not limited to, renal fibrosis, chronic obstructive pulmonary disease, poly-cystic ovary syndrome, restenosis, renovascular disease and organ transplant rejection; malignancies and diseases associated with neoangiogenesis (such as diabetic retinopathy); cancer, including, but not limited to, breast, ovary, colon, central nervous system, kidney and prostate cancers, and as imaging agents for the identification of metastatic cancers; myelofibrosis with myeloid metaplasia by regulating stromal cell hyperplasia and increases in extracellular matrix proteins; diabetic nephropathy and renal dialysis associated with nephropathy; septicemia, obesity, insulin resistance, proliferative diseases such as psoriasis, improving coagulation homeostasis, cerebrovascular disease, microvascular disease, hypertension, dementia, osteoporosis, asthma, and as a hormone replacement agent, treating, preventing or reversing progression of atherosclerosis, Alzheimer's disease, osteoporosis, osteopenia; reducing inflammatory markers, reducing C-reactive protein, or preventing or treating low grade vascular inflammation, stroke, dementia, coronary heart disease, primary and secondary prevention of myocardial infarction, stable and unstable angina, primary prevention of coronary events, secondary prevention of cardiovascular events, peripheral vascular disease, peripheral arterial disease, acute vascular syndromes, reducing the risk of undergoing a myocardial revascularization procedure, microvascular disease such as nephropathy, neuropathy, retinopathy and nephrotic syndrome, hypertension, Type 1 and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improving coagulation homeostasis, and/or improving endothelial function, and all forms of cerebrovascular diseases; septicemia, obesity, insulin resistance, psoriasis and related conditions, cerebrovascular diseases, arthritis, heart failure, angina and other cardiac conditions, malignant and premalignant lesions, and for topical wound healing and prevention of scarring.

When the disease is cancer, it can be leukemia, non-small cell lung cancer, colon cancer, central nervous system cancers, melanoma, kidney cancer, ovarian cancer, renal cancer, prostate cancer or breast cancer.

The invention furthermore relates to the use of the compounds according to the invention for the preparation of medicaments which are used in the treatment or prevention of the above mentioned diseases.

The compounds of the invention may be used in conjunction with procedures involving blood vessels, including vascular surgery, vascular graft and stent patency, organ, tissue and cell implantation and transplantation. The compounds in the invention may also be useful in the treatment of inflammatory diseases, septic shock and the vascular damage associated with infections.

The compounds of the invention are useful for the treatment of blood and blood products used in dialysis, blood storage in the fluid phase, especially ex vivo platelet aggregation. The present compounds may also be added to human plasma during the analysis of blood chemistry in hospital settings to determine the fibrinolytic capacity thereof.

The compounds in the present invention may also be used in combination with prothrombolytic, fibrinolytic and anti-coagulant agents.

The compounds of the invention may also be used in conjunction with protease inhibitor-containing highly active antiretroviral therapy (HAART) for the treatment of diseases which originate from fibrinolytic impairment and hypercoaguability of HIV-1 infected patients receiving such therapy.

A pharmaceutically effective amount of a compound herein refers to an amount of the compound which will inhibit the serine protease inhibitor PAI-1 in the mammal in need thereof to a sufficient extent to provide a desirable improvement in the condition in question or provide sufficient inhibition of the serine protease inhibitor PAI-1 to prevent, inhibit or limit the onset of the physiological basis for the malady or condition in question.

Compounds of the present invention may be used to treat diseases associated with elevated levels of PAI-1 as well as reduced levels of targets of PAI-1. For example, such diseases include but are not limited to pulmonary and cardiovascular diseases; cancers, including breast, lung, and ovarian cancers; Alzheimer's disease; and prophylaxis for prevention of the above conditions and others associated with increased PAI-1 activity or levels in vivo.

Compounds of the invention may be administered to mammalian patients in need of treatment or prophylaxis, in a therapeutically effective amount, consisting of an amount sufficient to provide prophylaxis or treatment of the disease condition in question. Compounds of the invention may be administered alone or in combination with one or more pharmaceutically acceptable carriers, excipients, other medicinally active components, and other pharmaceutically acceptable substances. Compounds of the invention may be administered orally or by injection, either intramuscular or intravenous.

### FIGURES

FIGS. 1 through 3 are graphs of PAI-1 bound to vitronectin versus concentration in log μM of compounds Q012110 and Q012059, as well as control compound, curcumin.

FIGS. 4A-4I represent dose response curves for anticancer activities of Q012052.

FIGS. 5A-5I represent dose response curves for anticancer activities of Q012132.

FIGS. 6A-6I represent dose response curves for anticancer activities of Q012135T.

FIGS. 7A-7I represent dose response curves for anticancer activities of Q012145T.

FIGS. 8A-8I represent dose response curves for anticancer activities of Q012147T.

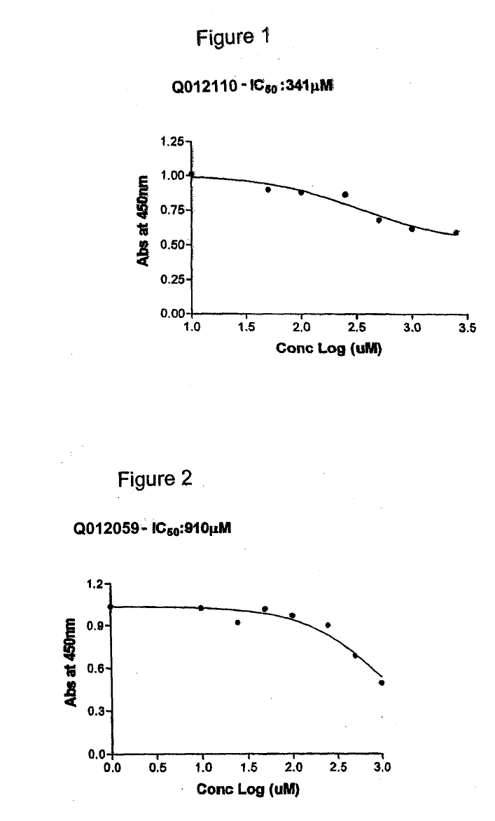

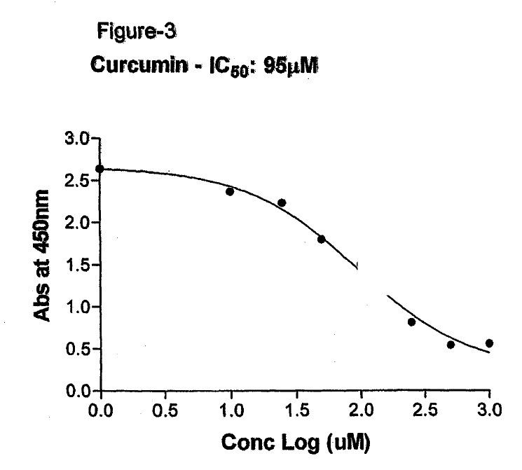

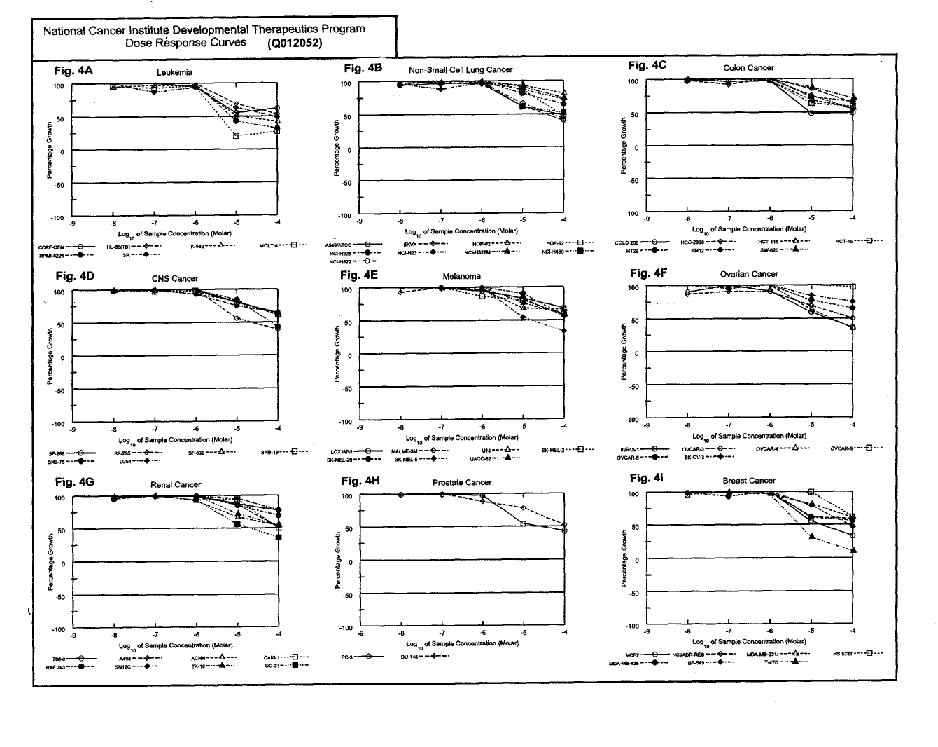

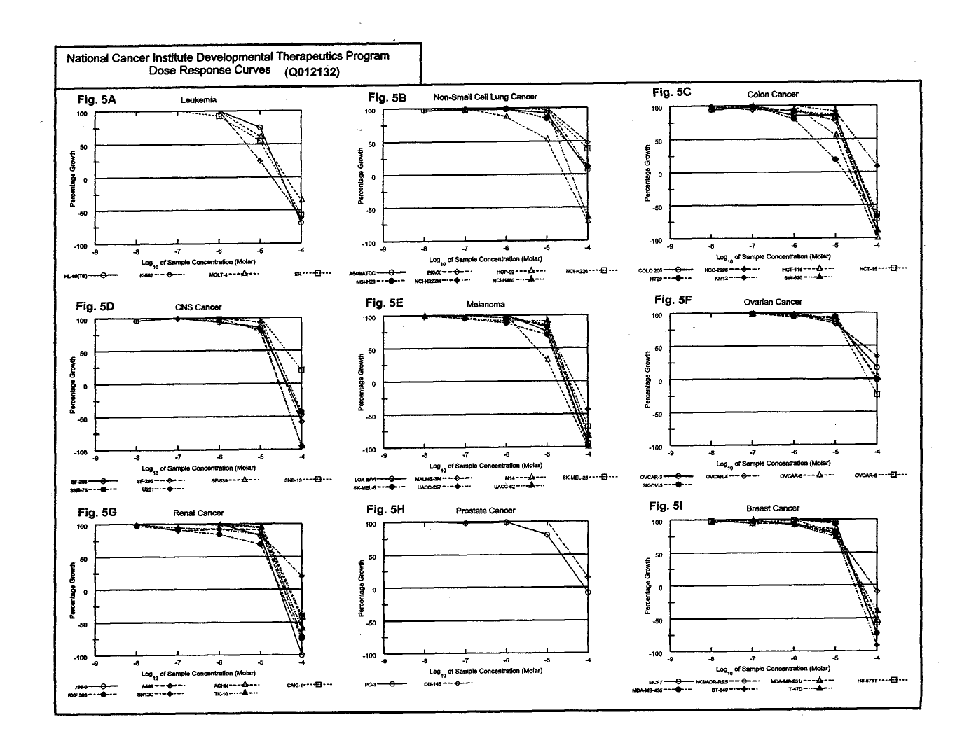

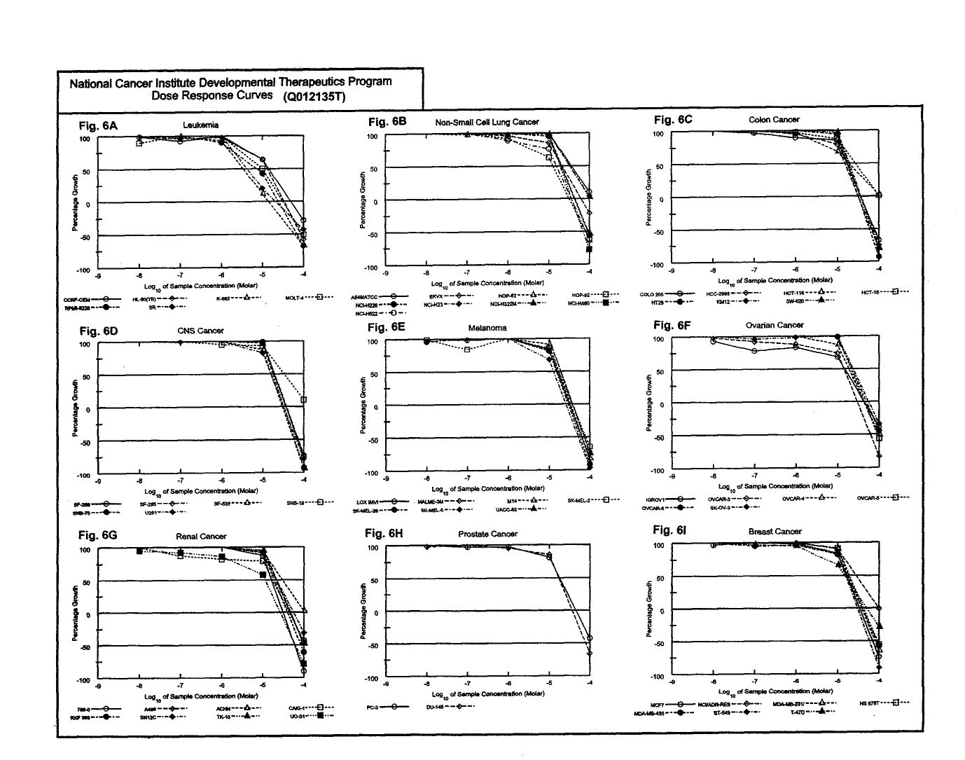

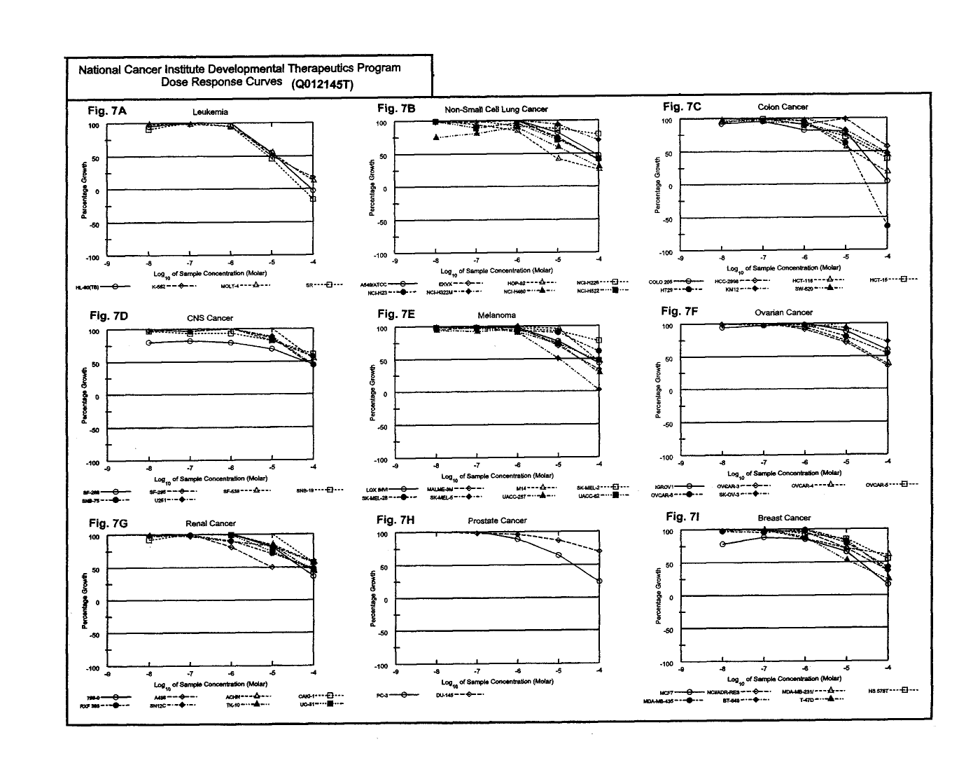

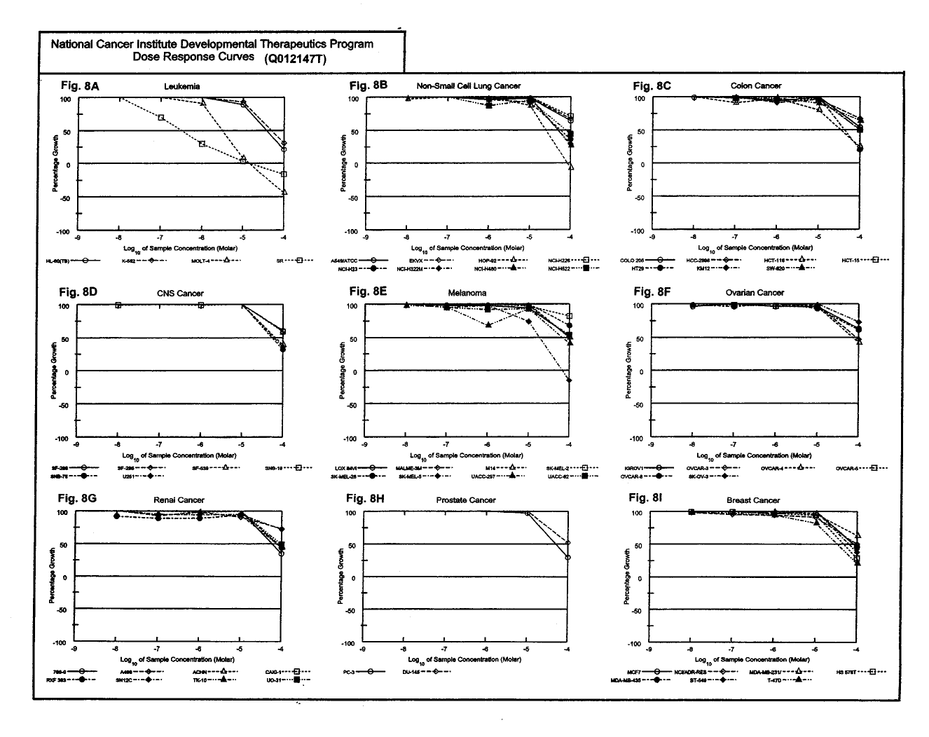

### DETAILED DESCRIPTION

Compounds according to the invention may be synthesized as follows:

### Example 1

### 2-Methoxy-4-{[(1-naphthylmethyl)amino]methyl}-6-[(4-pyrimidin-2-ylpiperazin-1-yl)methyl]phenol (Q012112)

A solution of 4-hydroxy-3-methoxy-5-[(4-pyrimidin-2-ylpiperazin-1-yl)methyl]benzaldehyde (412 mg, 1.24 mM) and 1-naphthylmethylamine (195 mg, 1.24 mM) in dry benzene (40 ml) was refluxed for 7 hrs using a Dean-Stark apparatus. The solvent was removed under vacuum and the residue was dissolved in abs. ethanol (15 ml). Sodium borohydride (150 mg, 3.9 mM) was added and the reaction mixture was stirred at r.t. for 36 hrs. The solvent was removed under vacuum, the residue was stirred with chloroform (50 ml), filtered and washed with chloroform. The filtrate was concentrated under vacuum and applied on a silica gel column run with hexane:ethyl acetate (gradient ethyl acetate from 0% to 50%) to give pure 2-methoxy-4-{[(1-naphthylmethyl)amino]methyl}-6-{4-[2-pyrimidylpiperazine]methyl}phenol as a colorless solid (290 mg, 50%). ¹H-NMR (CDCl₃): 8.31 (d, J=4.8 Hz, 2H), 8.04-8.10 (m, 1H), 7.83-7.90 (m, 1H), 7.78 (d, J=8.4 Hz, 1H), 7.40-7.55 (m, 4H), 6.88 (d, J=1.7 Hz, 1H), 6.02 (br.s, 1H), 5.91 (t, J=4.8 Hz, 1H), 4.24 (s, 2H), 3.90 (br.s, 6H), 3.83 (s, 2H), 3.76 (s, 2H), 2.62 (br.s, 4H).

### Example 2

### 2-Methoxy-4-({3-[(1H-imidazol-1-yl)propyl]amino}methyl)-6-[(4-pyrimidin-2-ylpiperazin-1-yl)methyl]phenol (Q012110)

A solution of 4-hydroxy-3-methoxy-5-[4-(2-pyrimidyl)piperazyl]methyl-benzaldehyde (332 mg, 1 mM) and 1-(3-aminopropyl)imidazole (125 mg, 1 mM) in dry 1,2-dichloroethane (10 ml) containing 4-methylmorpholine (0.5 ml) and anhydrous MgSO₄ (1.5 g) was stirred at r.t. for 1.5 hrs. Sodium triacetoxyborohydride ((650 mg, 3.2 mM) was added and stirring was continued for 48 hrs. Water (30 ml) and ethyl acetate (150 ml) were added, the organic layer was separated, washed with water and dried over MgSO₄. The solvent was removed under vacuum and the residue was chromatographed on a silica gel column using chloroform:EtOH (gradient EtOH from 0 to 6%) to give 2-methoxy-4-({3-[(1H-imidazol-1-yl)propyl]amino}methyl)-6-[(4-pyrimidin-2-ylpiperazin-1-yl)methyl]phenol (87 mg, 20%) as a colorless solid. ¹H-NMR (CDCl₃): 8.31 (d, J=4.8 Hz, 2H), 7.36 (br.s., 1H), 7.01 (br.s., 1H), 6.85 (br.s., 1H), 6.72 (br.s., 1H), 6.55 (br.s., 1H), 6.52 (t, J=4.8 Hz, 1H), 3.84-3.98 (br.s., 6H), 3.93 (s, 3H), 3.75 (s, 2H), 3.47 (br.s., 2H), 2.64 (br.s, 4H), 2.42-2.52 (m, 2H), 1.88-1.98 (m, 2H).

### Example 3

### (S)—N-[(4-methyl-2-oxo-2H-chromen-7-yl)oxyacetyl]-3-(thianaphthen-3-yl)alanine ethyl ester (Q012055)

To a solution of [(4-methyl-2-oxo-2H-chromen-7-yl)oxy]acetic acid (145 mg, 0.63 mM) in dry dichloromethane (15 ml) were added PyBOP (330 mg, 0.63 mM) and DIEA (250 mg, 1.95 mM). After stirring for 10 min at r.t. (S)-3-(thianaphthen-3-yl)alanine ethyl ester hydrochloride (180 mg, 0.63 mM) was added in one portion and the reaction mixture was stirred at r.t. for 3 hrs. The solvent was removed under vacuum and the residue was purified by column chromatography on silica gel with hexane:ethyl acetate=50:50 to give (S)—N-[(4-methyl-2-oxo-2H-chromen-7-yl)oxyacetyl]-3-(thianaphthen-3-yl)alanine ethyl ester as a colorless oil (295 mg, 100%). ¹H-NMR (CDCl₃): 7.80-7.88 (m, 1H), 7.74-7.80 (m, 1H), 7.47 (d, J=8.8 Hz, 1H), 7.30-7.40 (m, 2H), 7.13 (s, 1H), 7.04 (d, J=7.6 Hz, 1H), 6.74 (dd, J=8.8, 2.6 Hz, 1H), 6.73 (s, 1H), 5.04 (q, J=7.8 Hz, 1H), 4.51 (d, J=9.2 Hz, 1H), 4.48 (d, J=9.2 Hz, 1H), 4.20-4.40 (m, 2H), 3.45 (dd, J=15.0, 6.0 Hz, 1H), 3.41 (dd, J=15.0, 6.0 Hz, 1H), 2.39 (s, 3H).

### Example 4

### (2S)-2-({[(4-Methyl-2-oxo-2H-chromen-7-yl)amino]carbonyl}amino)-3-phenylpropanoic acid ethyl ester (Q012053)

To a solution of triphosgene (165 mg, 0.55 mM) in dry dichloromethane (3 ml) was added dropwise a solution of (S)-phenylalanine ethyl ester hydrochloride (342 mg, 1.5 mM) and DIEA (430 mg, 3.3 mM) in dry dichloromethane (3 ml) over 30 min. The reaction mixture was stirred for 10 min at r.t. and a solution of 7-amino-4-methylcoumarin (262 mg, 1.5 mM) and DIEA (430 mg, 3,3 mM) in dry dichloromethane (3 ml) was added, followed by the addition of dry DMF (2 ml). After stirring for 1 h at r.t. the solvents were removed under vacuum, the residue was dissolved in ethyl acetate (250 ml). The organic solution was washed with 1 N HCl (120 ml) and then with water (4×125 ml). Drying with MgSO₄ and evaporation of the solvent under vacuum gave a residue which was stirred with diisopropyl ether (5 ml) for 3 hrs, filtered and washed with diisopropyl ether to give pure (2S)-2-({[(4-methyl-2-oxo-2H-chromen-7-yl)amino]carbonyl}amino)-3-phenylpropanoic acid ethyl ester (66 mg, 12%) as a colorless solid. ¹H-NMR (DMSO-d₆): 9.21 (s, 1H), 7.62 (d, J=8.8 Hz, 1H), 7.53 (d, J=1.8 Hz, 1H), 7.18-7.34 (m, 5H), 6.65 (d, J=8.0 Hz, 1H), 6.18 (br.s, 1H), 4.52 (dd, J=13.4, 6.4 Hz, 1H), 4.09 (q, J=7.4 Hz, 2H), 3.07 (dd, J=18.0, 6.4 Hz, 1H), 3.00 (dd, J=18.0, 13.4 Hz, 1H), 2.36 (s, 3H), 1.15 (t, J=7.4 Hz, 3H).

### Example 5

### tert-Butyl 4-hydroxy-3-{[(2-hydroxyethyl)amino]carbonyl}phenylcarbamate (Q012021B)

To a solution of 5-(BOC-amino)salicylic acid (7.59 g, 30 mM) and N-hydroxysuccinimide (3.7 g, 32 mM) in dry dioxane (100 ml) cooled to 0° C. was added 1,3-dicyclohexylcarbodiimide (6.6 g, 32 mM) in five portions over 30 min. The cooling bath was removed and the reaction mixture was stirred at r.t. for 24 hrs. 2-Hydroxyethylamine (2.0 g, 33 mM) was added and the reaction mixture was stirred at r.t. for 3 days. Ethyl acetate was added and the N,N′-dicyclohexyl-urea was filtered off and washed with ethyl acetate (2×200 ml). The filtrate was washed with water, dried over MgSO₄, filtered and the solvents were removed under vacuum. The residue was crystallized from ethyl acetate (45 ml) and hexane (75 ml) to give tert-butyl 4-hydroxy-3-{[(2-hydroxyethyl)amino]carbonyl}phenylcarbamate (7.61 g, 86%) as a colorless solid. ¹H-NMR (DMSO-d₆): 11.71 (s, 1H), 9.10 (s, 1H), 8.67 (t, J=5.4 Hz, 1H), 7.89 (d, J=2 Hz, 1H), 7.29 (dd, J=8.9, 2.0 Hz, 1H), 6.81 (d, J=8.9 Hz, 1H), 4.79 (t, J=5.1 Hz, 1H), 3.50 (q, J=6.0 Hz, 2H), 3.34 (q, J=5.4 Hz, 2H).

### Example 6

### {2-[5-(BOC-amino)-2-hydroxybenzoylamino]}ethyl 4-nitrophenyl carbonate (Q012051)

To a solution of tert-Butyl 4-hydroxy-3-{[(2-hydroxyethyl)amino]carbonyl}phenylcarbamate (2.33 g, 7.9 mM) in dry pyridine (16 ml) was added 4-nitrophenyl-chloroformate (1.64 g, 8.1 mM) and the reaction mixture was stirred at r.t. for 20 hrs. The solvent was removed under vacuum and the residue was applied on a silica gel column run with hexane:ethyl acetate (gradient ethyl acetate from 15% to 25%) to give {2-[5-(BOC-amino)-2-hydroxybenzoylamino]}ethyl 4-nitrophenyl carbonate (650 mg, containing ca. 50% of 4-nitrophenol) as a yellow solid. ¹H-NMR (CDCl₃): 8.65 (s, 1H), 8.26 (d, J=9.0 Hz, 2H), 7.40 (d, J=9.0 Hz, 2H), 8.04 (d, J=2.5 Hz, 1H), 7.81 (dd, J=9.0, 2.5 Hz, 1H), 7.24 (d, J=9.0 Hz, 1H), 6.85 (br.d, J=4.5 Hz, 1H), 4.50 (t, J=5.5 Hz, 2H), 4.47 (t, J=5.5 Hz, 2H), 1.55 (s, 9H).

### Example 7

### 2-[5-(BOC-amino)-2-hydroxybenzoylamino]ethyl 2-[2,5-bis-(benzyl-oxy)benzoylamino]ethylcarbamate (Q012052)

A solution of {2-[5-(BOC-amino)-2-hydroxy-benzoylamino]}ethyl 4-nitrophenyl carbonate (380 mg, 0.7 mM) and 2-[2,5-bis-(benzyloxy)benzoylamino]ethylamine (265 mg, 0.7 mM) in dry DMF (3 ml) was stirred at r.t. for 20 hrs. The solvent was removed under vacuum and the residue was crystallized from ethyl acetate (5 ml) and diisopropyl ether (5 ml) to give pure 2-[5-(BOC-amino)-2-hydroxy-benzoylamino]ethyl 2-[2,5-bis-(benzyloxy)benzoylamino]ethylcarbamate (500 mg, 100%) as a colorless solid. ¹H-NMR (CDCl₃): 8.22 (t, J=4.8 Hz, 1H), 7.91 (d, J=9.8 Hz, 1H), 7.83 (d, J=3.0 Hz, 1H), 7.75 (d, J=1.8 Hz, 1H), 7.30-7.45 (m, 10H), 7.17 (d, J=9.0 Hz, 1H), 7.04 (dd, J=9.0, 3.0 Hz, 1H), 6.96 (d, J=9.0 Hz, 1H), 6.76 (s, 1H), 5.14 (s, 2H), 5.07 (s, 2H), 4.90 (br.s, 1H), 4.32 (t, J=4.5 Hz, 2H), 4.27 (t, J=4.5 Hz, 2H), 3.45 (q, J=5.4 Hz, 2H), 3.20 (q, J=5.4 Hz, 2H), 1.52 (s, 9H).

### Example 8

### (S)—N-[(4-methyl-2-oxo-2H-chromen-7-yl)oxyacetyl]-3-(thianaphthen-3-yl)alanine (Q012055A)

A suspension of (S)—N-[(4-methyl-2-oxo-2H-chromen-7-yl)oxyacetyl]-3-(thianaphthen-3-yl)alanine ethyl ester (320 mg, 0.69 mM) in methanol (6 ml) and water (2 ml) containing NaOH (160 mg, 4 mM) was stirred at r.t for 4 hrs. The clear solution was rotavaped down to ca. 1.5 ml, cooled in an ice bath and acidified to pH=2 with 6 N HCl. The mixture was filtered, the solid was washed with cold water and dried under vacuum at r.t. to provide pure (S)—N-[(4-methyl-2-oxo-2H-chromen-7-yl)oxyacetyl]-3-(thianaphthen-3-yl)alanine (208 mg, 70%) as a colorless solid. ¹H-NMR (DMSO-d₆): 8.48 (d, J=7.5 Hz, 1H), 7.93 (d, J=7.4 Hz, 1H), 7.83 (d, J=7.2 Hz, 1H), 7.64 (d, J=8.4 Hz, 1H), 7.30-7.42 (m, 3H), 6.90 (dd, J=9.0, 2.4 Hz, 1H), 6.88 (s, 1H), 6.23 (q, J=0.9 Hz, 1H), 4.62 (d, J=15.0 Hz, 1H), 4.58 (d, J=15.0 Hz, 1H), 3.24 (dd, J=15.0, 9.0 Hz, 1H), 2.39 (d, J=0.9 Hz, 3H).

### Example 9

### (S)—N-(5-bromonicotinoyl)-3-(thianaphthen-3-yl)alanine hydrochloride (Q011265)

To a stirred suspension of (S)-3-(thianaphthen-3-yl)alanine hydrochloride (1.00 g, 3.89 mM) in 40 ml of water under nitrogen was cooled to 0° C., and pH=12.2 was adjusted using 2 N NaOH. A solution of 5-bromo-nicotinoylchloride in dry toluene (6 ml) was added dropwise at 0° C. over ca. 50 min maintaining pH=11.2 by addition of 2N NaOH. After stirring for 2 hrs at 0° C. the reaction mixture was extracted with diethyl ether (3×40 ml), and pH=2.0 was adjusted using 6N HCl. The resulting paste was stirred at 0° C. for 10 min, filtered, the solid was washed with cold water (6×25 ml) and dried under vacuum at r.t. to give (S)—N-(5-bromonicotinoyl)-3-(thianaphthen-3-yl)alanine hydrochloride (1.53 g, 90%) as a colorless solid. ¹H-NMR (CD₃OD): 8.77 (d, J=2.0 Hz, 1H), 8.76 (d, J=2.8 Hz, 1H), 8.22 (t, J=2.2 Hz, 1H), 7.87 (br.t, J=6.4 Hz, 2H), 7.30-7.42 (m, 3H), 5.00 (dd, J=9.5, 4.5 Hz, 1H), 3.52 (dd, J=14.2, 4.5 Hz, 1H), 3.48 (dd, J=14.2, 9.5 Hz, 1H).

### Example 10

### (2S)-2-[({[(1S)-1-carboxy-2-(thianaphthen-3-yl)ethyl]amino}carbonyl)amino]-5-({imino[(4-methoxy-2,3,6-trimethylphenylsulfonyl)amino]methyl}amino)pentanoic acid diethyl ester (Q012059)

Triphosgene (60 mg, 0.19 mM) was dissolved in dry dichloromethane (4 ml). A solution of (S)-3-(thianaphthen-3-yl)alanine ethyl ester hydrochloride (142 mg, 0.5 mM) and DIEA (140 mg, 1.1 mM) in dry dichloromethane (4 ml) was added dropwise over 20 min.

Stirring was continued for 20 min and a solution of (S)-2-amino-5-({imino[(4-methoxy-2,3,6-trimethyl-phenylsulfonyl)amino]methyl}amino)pentanoic acid ethyl ester hydrochloride (351 mg, 1 mM) and DIEA (140 mg, 1.1 mM) in dry dichloromethane (4 ml) was added dropwise over 20 min. Three hours later the solvents were removed under vacuum, the residue was dissolved in ethyl acetate (200 ml), washed with cold 2N HCl, then with cold water, dried over MgSO₄ and the solvent was removed under vacuum. The residue was applied on Sephadex LH-20™ column run with methanol to give the target compound (83 mg, 12%) as a colorless oil. ¹H-NMR (CDCl₃): 7.86 (br.d, J=8.0 Hz, 1H), 7.78 (br.d, J=8.0 Hz, 1H), 7.40 (br.t, J=8.0 Hz, 1H), 7.35 (br.t, J=8.0 Hz, 1H), 7.18 (s, 1H), 6.51 (s, 1H), 6.23 (s, 2H), 5.57 (d, J=7.5 Hz, 1H), 5.30 (br.dd, J=7.1, 8.0 Hz, 1H), 4.72 (dd, J=12.0, 7.1 Hz, 1H), 4.0-4.20 (m, 4H), 3.82 (s, 3H), 3.30 (dd, J=15.0, 5.0 Hz, 1H), 3.35 (dd, J=15.0, 8.2 Hz, 1H), 3.10-3.30 (m, 2H), 2.67 (s, 3H), 2.60 (s, 3H), 2.12 (s, 3H), 1.50-1.70 (m, 4H), 1.25 (t, J=7.2 Hz, 3H), 1.15 (t, J=7.1 Hz, 3H).

### Example 11

### 5-[(Z)-(1-benzyl-2,5-dioxoimidazolidin-4-ylidene)methyl]-2-hydroxybenzoic acid (Q013012)

A suspension of 5-formyl-2-hydroxybenzoic acid (332 mg, 2 mM), 3-benzylimidazolidine-2,4-dione (380 mg, 2 mM) and 2-aminoethanol (360 mg, 6 mM) in water (10 ml) was refluxed for 4 hrs. The reaction mixture was cooled to r.t., acidified to pH=2 with 6N HCl, filtered and the solid was washed with water and dried under vacuum to afford 5-[(Z)-(1-benzyl-2,5-dioxoimidazolidin-4-ylidene)methyl]-2-hydroxybenzoic acid (167 mg, 70%) as a colorless solid. ¹H-NMR (DMSO-d₆): 10.88 (s, 1H), 7.99 d, J=2.4 Hz, 1H), 7.81 (dd, J=8.5, 2.4 Hz, 1H), 7.2-7.4 (m, 5H), 6.98 (d, J=8.5 Hz, 1H), 6.56 (s, 1H), 4.65 (s, 2H).

The invention also provides for the compounds listed below:
2-Methoxy-4-({[2-(pyridin-2-yl)ethyl]amino}methyl)-6-[(4-pyridin-2-ylpiperazin-1-yl)methyl]phenol (Q012119T)
2-Methoxy-4-({[(piperidin-4-yl)methyl]amino}methyl)-6-[(4-pyridin-2-ylpiperazin-1-yl)methyl]phenol (Q012131)
2-Methoxy-4-({[(N-t-butyloxycarbonylpiperidin-4-yl)methyl]amino}methyl)-6-[(4-pyridin-2-ylpiperazin-1-yl)methyl]phenol (Q012132)
(2S)-3-(thianaphthen-3-yl)-2-({[(3R)-piperidin-3-yl]carbonyl}amino)propanoic acid (Q012129)
(2S)-3-(thianaphthen-3-yl)-2-({[(3R)-piperidin-3-yl]carbonyl}amino)propanoic acid ethyl ester (Q012127)
(2S)-3-(thianaphthen-3-yl)-2-({[(3R)—N-t-butyloxycarbonylpiperidin-3-yl]carbonyl}amino)propanoic acid ethyl ester (Q012126)
(2S)-3-(thianaphthen-3-yl)-2-({[(3R)—N-t-butyloxycarbonylpiperidin-3-yl]carbonyl}amino)propanoic acid (Q012128)
(2S)-3-(thianaphthen-3-yl)-2-({[(3R)—N-[amino(imino)methyl]piperidin-3-yl]carbonyl}amino)propionic acid (Q012130)
(2S)-2-({[2-oxo-4-(trifluoromethyl)-2H-chromen-7-yl]oxy}acetyl)amino-3-(thianaphthen-3-yl)propanoic acid (Q012125)
(2S)-2-({[2-oxo-4-(trifluoromethyl)-2H-chromen-7-yl]oxy}acetyl)amino-3-(thianaphthen-3-yl)propanoic acid ethyl ester (Q012124)

Compounds according to the invention were tested for inhibition of PAI-1 activity by assays which measured the inhibitory activity of PAI-1 towards uPA as well as PAI-1's interaction with vitronectin. Since PAI-1 is also a key regulator of tumorogenesis and metastasis¹, compounds according to the invention were also screened by the National Cancer Institute (NCI) for anti-cancer activity in 60 different human cancer cell lines.

### PAI-1/uPA Assay

Principle: The assay is based on using a fluorescent substrate (SpectroFluor) to measure residual uPA following inhibition of uPA by PAI-1. PAI-1 used in the assay is pre-treated in the presence of increasing concentrations of the lead compounds to screen for any inhibitory effects on PAI-1.

Method: This assay was performed in 96 well microtiter plates. The concentrations of uPA (8 nM) and PAI-1 (6 nM) used in this assay were carefully optimized when using the fluorescent substrate, SpectroFluor™. Stock solutions (10 mM) of all lead compounds were prepared fresh in DMSO. Further desired dilutions of all lead compounds was also done in DMSO but the concentration of DMSO in the PAI-1 pre-treatment or in the final assay volume did not exceed 10%. 20 μl of 6 nM wildtype human PAI-1 (Molecular Innovations, Lot# HWT-804) was incubated for 15-20 minutes at room temperature in 96 well microtiter plates either with buffer, (50 mM HEPES, 150 mM NaCl, 0.05% Tween-20, 1% BSA, pH 6.6) or various concentrations (10 nM to 100 μM final concentration) of lead compounds in a total volume of 80 μl. A 20 μl aliquot of 8 nM uPA (HMW) solution in 0.05M Tris Buffered™ saline (TBS), pH 8.4 was added to each well and the mixture incubated for an additional 5 minutes at RT. The proteolytic reaction was initiated by addition of 50 μl of 0.1 mM SpectroFluor (Spectrozyme UK Fluorogenic substrate) to each well.

The kinetics of the free chromophore, 7-amino-4-methylcoumarin (AMC) released in the course of peptide cleavage by the protease, uPA, was monitored for 30 minutes at 37° C. on a spectrofluorometric microplate reader with an excitation wavelength of 360 nm and emission wavelength of 440 nm. Kinetic data regarding the rate of substrate cleavage by uPA was acquired with Softmax PrO™ software.

The difference between the residual uPA activity in the presence or absence of untreated PAI-1, at the molar ratio used, was considered as the “total” PAI-1 activity (100%). The inhibition of uPA by PAI-1 treated with lead compounds was expressed as a percentage of the initial activity of untreated PAI-1. Controls were performed during each experiment to ensure the nearly complete inhibition of uPA by untreated PAI-1 and also to ensure the absence of any direct effect of the lead compounds on uPA alone or on SpectroFluor.

Results: uPA inhibition serves as a functional index of PAI-1 inhibition by these small molecule compounds, possibly by direct binding to the PAI-1 molecule and subsequent structural/conformational alterations. As shown in Table 1, different lead compounds tested had varying ability to inhibit PAI-1 with respect to its interaction with uPA. However, in this in vitro assay, the maximal inhibition of PAI-1 achieved by these compounds was in the range of 8-57%, within the concentration range tested.

### Activity of Compounds in Inhibiting PAI-1 and Vitronectin Interaction

Vitronectin, an extra-cellular matrix protein, can bind and stabilize PAI-1 in active form, enhancing, prolonging and localizing its activity. Compounds according to the invention were studied for inhibiting PAI-1's interaction with vitronectin.

Principle: This ELISA assay is based on the interaction of wildtype human PAI-1 (aa 110-145) with the N-terminal somatomedin B-like (SMB) domain (aa 1-40) on vitronectin. PAI-1 was allowed to bind to vitronectin coated plates. The bound PAI-1 was detected with a HRP-conjugated polyclonal goat anti-human PAI-1 antibody using a standard ELISA method. In order to screen these compounds for their ability to disrupt PAI-1's interaction with vitronectin, PAI-1 was pre-incubated with various concentrations of the lead compounds before being allowed to bind to vitronectin.

Method: Human monomeric vitronectin (Vn; Molecular Innovations) was diluted to a concentration of 2.5 μg/ml in a buffer containing 100 mM Na₂CO₃, pH 9.6. 96-well flat-bottom microtiter immunoassay plates (Immulon 4HBX #6508) were coated by adding 50 μl per well of vitronectin and incubating for 16 hours at 4° C. After three washes with 100 mM PBS containing 0.05% Tween-20 (v/v), the wells were blocked by addition of 200 μL per well of Pierce Super-block™ (Product #37515) and incubated at room temperature for 30 minutes. The wells were again washed three times with 10 mM PBS containing 0.05% Tween-20, drained and blot dried. After air drying at room temperature for 4 to 5 hours, the plates were individually sealed in Kapak™ laminated foil pouches (5×8 inch) with 2×0.5 g dessicant packs and stored at 2-8° C. until use.

In a separate 96-well microtiter plate 20 μL per well of wildtype human PAI-1 (15 nM final concentration) was added with or without various concentrations of lead compounds (1 μM to 1000 μM final concentration) and incubated at room temperature for 15 minutes on a rotating shaker. This PAI-1/compound mixture was then transferred to a vitronectin coated plate and allowed to incubate for 1 hour at room temperature on a rotating shaker. The plate was then washed three times with 10 mM PBS containing 0.05% Tween-20™. 50 μl per well of detection antibody (HRP-conjugated polyclonal goat anti-human PAI-1 antibody) diluted 1:100 was added and allowed to incubate at RT on a rotating shaker for an additional 1 hour. After a subsequent 3× wash, binding of PAI-1 to the solid phase was visualized by adding 100 μL per well of the HRP substrate, TMB. The reaction was stopped after color development with 50 μL per well of 0.5 M H₂SO₄. Optical density was measured at 450 nm on a SoftMax PrO™ plate reader.

Where vitronectin inhibition was evident, IC₅₀ values for a test compound were calculated by fitting a non-linear sigmoidal regression curve to log micromolar concentration versus optical density data.

Results of the PAI-1 vitronectin interaction assay: Compounds of the invention generally showed low inhibitory activities towards PAI-1 interaction with vitronectin within the concentration range tested. Compounds Q012110 and Q012059 inhibited PAI-1-vitronectin interaction with IC₅₀ values of 340 and 910 μM, respectively. These data suggest that compounds of the invention may not directly block the PAI-1 site (encompassing amino-acids 110-145) required for high-affinity vitronectin binding. Although the therapeutic value of this particular characteristic of these compounds remains to be delineated, several compounds of this invention appear to selectively inhibit PAI-1's inhibition of the protease uPA without affecting PAI-1's ability to interact with vitronectin.

### National Cancer Institute Anticancer Tests

Principle: The National Cancer Institute (NCI) in vitro anticancer screen comprises of a panel of 60 different human cancer cell-lines representing leukemia, melanoma and cancers of the lung, colon, brain, ovary, kidney, prostate and breast. Each cell line was exposed to five different log concentrations of the compounds according to the invention, and effects on cell density and proliferation were graphed to obtain the pharmacokinetic concentration parameters GI50, TGI and LC50 which are a measure of a compound's ability to inhibit growth and proliferation (cytostatic) or to kill the cancer cells (cytotoxic).

Method: The 60 cell lines used in the NCI anticancer screening procedure have been described²⁷ and the screen itself has been extensively established.²⁸ Briefly, cell suspensions of the human cancer cell lines are diluted according to the particular cell type and their expected target cell density (5000-40,000 cells per well). 100 μL of the cell suspension is added to each well in a 96-well microtiter plate. Inoculates are allowed a preincubation period of 24 h at 37° C. for attachment and stabilization. Dilutions of the test compound at twice the intended concentration (in DMSO) are added at time zero (T0) in 100 μL aliquots to the microtiter plate wells. Usually, the test compounds are evaluated in five dilutions ranging from 10 nM to 100 μM. Incubations are carried out for 48 h in 5% CO₂ and 100% humidity. Cell proliferation is then measured by a protein stain assay using sulforhodamine B. An ELISA plate reader is used to obtain optical densities and the following special concentration parameters are calculated:

### Special Concentration Parameters GI50, TGI and LC50:

The GI50 value is a measure of the compound's ability to inhibit growth of cancer cells by 50% as compared to a “control” in the absence of the test compound. The NCI renamed the pharmacokinetic term IC50, the concentration that causes 50% growth inhibition, as the GI50 value since it incorporates a correction for the cell count at time zero when the cells are first added to the microtiter plate wells. The optical density of the test well of cancer cells after a 48 hour period of exposure to the compound is “T”, which is a factor in the parameter TGI (total growth inhibition) which measures a compound's cytostatic ability. The LC50 (half lethal concentration) is the concentration of a compound which kills 50% of the cancer cells, and represents a cytotoxic effect. The control optical density is not factored into the calculation of the LC50.

Results: Compounds according to the invention including Q012052, Q012132, Q012135T, Q012145T and Q012147T inhibited growth and proliferation of human primary cancer cell-lines representing various cancers. Tables 2 and 3 depict selected compounds from this invention with high anti-cancer potency in the NCI anti cancer in vitro screen. It can be seen that the compounds have distinct profiles with respect to their anticancer efficacy. At low micromolar concentrations, Q012132 and Q012135T had cytostatic and cytotoxic effects on human cancer cell-lines representing leukemia, melanoma and cancers of the colon and central nervous system (Table 3). Other compounds as depicted in Table 2 had mainly cytostatic effects with a preferred biological response pattern toward leukemias, melanomas, central nervous system, colon and breast cancers. Further selection, modification and testing of lead compounds with a view towards enhancing their PAI-1 inhibitory activity and anticancer potential may be possible. Since the compounds in this invention are generally of low toxicity, micromolar concentrations in vivo are certainly achievable pharmaceutically.

TABLE 1
Bioactivity assay of PAI-1 interaction with uPA

| | Conc. of | | |
|---|---|---|---|
| Name of the | compound required | | Percent PAI-1 |
| Compound | for PAI-1 inhibition | | inhibition |
| Q012110 | 5 | μM | 57 |
| Q012112H | 10 | μM | 33 |
| Q012119T | No inhibition | | . . . |
| Q012131H | 500 | μM | 15 |
| Q012132 | No inhibition | | . . . |
| Q012135T | 100 | μM | 8 |
| Q012136 | 50 | μM | 15 |
| Q012143 | 250 | μM | 22 |
| Q012145T | No inhibition | | . . . |
| Q012146 | No inhibition | | . . . |
| Q012147T | No inhibition | | . . . |
| Q012148 | No inhibition | | . . . |
| Q013024 | No inhibition | | . . . |
| Q012053 | 10 | μM | 44 |
| Q012053A | 10 | μM | 43 |
| Q012055 | 50 | nM | 51 |
| Q012055A | 1 | μM | 41 |
| Q012056 | 5 | μM | 44 |
| Q012056A | 100 | nM | 26 |
| Q012059 | 10 | μM | 23 |
| Q012059H | 100 | μM | 33 |
| Q011265 | 1 | μM | 20 |
| Q012124 | 50 | nM | 13 |
| Q012128 | 10 | μM | 14.5 |
| Q012129 | 1 | μM | 23 |
| Q012125 | No inhibition | | . . . |
| Q012126 | 50 | μM | 19 |
| Q012127 | 250 | μM | 13 |
| Q012130 | No inhibition | | . . . |
| Q012137 | 100 | μM | 45 |
| Q012138 | 100 | μM | 24 |
| Q012139 | No inhibition | | . . . |
| Q012140 | No inhibition | | . . . |
| Q012141 | No inhibition | | . . . |
| Q012052 | 50 | nM | 45 |
| Q012021B | 10 | μM | 25 |
| Q012038 | 10 | μM | 35 |
| Q013012 | No inhibition | | . . . |
| Q013013 | No inhibition | | . . . |
| Q013018 | No inhibition | | . . . |
| Q013019 | No inhibition | | . . . |
| Q013021 | 1 | μM | 25 |
| Curcumin | 500 | nM | 22 |

TABLE 2
Growth Inhibition (GI50) of Human Cancer Cell-Lines
by Compounds According to this Invention

| | Q012052 | Q012132 | Q012135T | Q012145T | Q012147T |
|---|---|---|---|---|---|
| Leukemia | | | | | |
| CCRF-CEM | >100 μM | — | 14.1 μM | — | — |
| HL-60(TB) | >100 μM | 14.8 μM | 13.1 μM | 12.4 μM | 37.5 μM |
| K-562 | 14.0 μM | 5.21 μM | 3.4 μM | 10.2 μM | 49.3 μM |
| MOLT-4 | 4.05 μM | 13.0 μM | 10.1 μM | 13.0 μM | 3.13 μM |
| RPMI-8226 | 7.58 μM | — | 7.14 μM | — | — |
| SR | 89.8 μM | 11.0 μM | 4.18 μM | 8.3 μM | 0.32 μM |
| Non-Small | | | | | |
| Cell Lung | | | | | |
| A549/ATCC | 68.8 μM | 31.2 μM | 36.9 μM | 84.3 μM | >100 μM |
| EKVX | 35.1 μM | 91.2 μM | 21.3 μM | 51.5 μM | >100 μM |
| HOP-62 | >100 μM | — | 20.6 μM | — | — |
| HOP-92 | 83.1 μM | 10.7 μM | 12.8 μM | 6.57 μM | 24.8 μM |
| NCI-H226 | >100 μM | 65.5 μM | 19.8 μM | >100 μM | >100 μM |
| NCI-H23 | >100 μM | 29.6 μM | 34.2 μM | 51.6 μM | 55.9 μM |
| NCI-H322M | >100 μM | 37.8 μM | 33.8 μM | >100 μM | 49.4 μM |
| NCI-H460 | >100 μM | 21.3 μM | 20.2 μM | 21.9 μM | 60.6 μM |
| NCI-H522 | 49.7 μM | | 15.5 μM | 52.9 μM | 76.3 μM |
| Colon | | | | | |
| COLO 205 | 8.97 μM | 16.8 μM | 17.0 μM | 24.8 μM | >100 μM |
| HCC-2998 | >100 μM | 14.5 μM | 15.3 μM | >100 μM | >100 μM |
| HCT-116 | >100 μM | 10.8 μM | 18.6 μM | 15.2 μM | 34.4 μM |
| HCT-15 | >100 μM | 16.8 μM | 26.9 μM | 46.4 μM | 98.3 μM |
| HT29 | >100 μM | 3.11 μM | 17.1 μM | 12.7 μM | 43.2 μM |
| KM12 | >100 μM | 31.6 μM | 21.0 μM | 80.1 μM | >100 μM |
| SW-620 | >100 μM | 16.1 μM | 18.5 μM | 63.7 μM | >100 μM |
| CNS | | | | | |
| SF-268 | >100 μM | 18.5 μM | 19.1 μM | 61.5 μM | >100 μM |
| SF-295 | 21.9 μM | 19.3 μM | 19.6 μM | 69.9 μM | >100 μM |
| SF-539 | >100 μM | 14.6 μM | 16.0 μM | >100 μM | 73.4 μM |
| SNB-19 | >100 μM | 41.8 μM | 33.1 μM | >100 μM | >100 μM |
| SNB-75 | 68.9 μM | 22.3 μM | 17.6 μM | >100 μM | 56.6 μM |
| U251 | >100 μM | 15.1 μM | 15.8 μM | 75.9 μM | 61.9 μM |
| Melanoma | | | | | |
| LOX IMVI | >100 μM | 14.1 μM | 15.8 μM | 60.4 μM | >100 μM |
| MALME- | >100 μM | 15.7 μM | 20.1 μM | 34.6 μM | 99.9 μM |
| 3M | | | | | |
| M14 | >100 μM | 5.8 μM | 17.2 μM | 83.3 μM | 99.0 μM |
| SK-MEL-2 | >100 μM | | 17.2 μM | >100 μM | >100 μM |
| SK-MEL-28 | >100 μM | 16.8 μM | 15.1 μM | >100 μM | >100 μM |
| SK-MEL-5 | >100 μM | 13.1 μM | 12.9 μM | 10.7 μM | 18.7 μM |
| UACC-257 | 15.6 μM | 18.4 μM | — | 33.0 μM | 67.3 μM |
| UACC-62 | >100 μM | 17.2 μM | 20.1 μM | 74.5 μM | >100 μM |
| Ovarian | | | | | |
| IGR-OV1 | 25.3 μM | — | 14.7 μM | >100 μM | >100 μM |
| OVCAR-3 | >100 μM | 33.1 μM | 14.1 μM | 37.5 μM | 86.2 μM |
| OVCAR-4 | 29.1 μM | 47.8 μM | 18.6 μM | 46.9 μM | 70.2 μM |
| OVCAR-5 | >100 μM | 29.3 μM | 23.4 μM | >100 μM | >100 μM |
| OVCAR-8 | >100 μM | 22.3 μM | 22.1 μM | >100 μM | >100 μM |
| SK-OV-3 | >100 μM | 29.4 μM | 24.9 μM | >100 μM | >100 μM |
| Renal | | | | | |
| 786-0 | >100 μM | 14.9 μM | 16.2 μM | 48.6 μM | 60.2 μM |
| A498 | >100 μM | 17.8 μM | 25.8 μM | 16.2 μM | >100 μM |
| ACHN | >100 μM | 21.0 μM | 29.9 μM | >100 μM | 78.2 μM |
| CAKI-1 | 98.0 μM | 22.8 μM | 17.2 μM | >100 μM | >100 μM |
| RXF 393 | >100 μM | 13.6 μM | 18.8 μM | 80.3 μM | 76.5 μM |
| SN12C | >100 μM | 34.0 μM | 21.7 μM | >100 μM | >100 μM |
| TK-10 | >100 μM | 19.5 μM | 20.6 μM | 70.4 μM | 83.0 μM |
| UO-31 | 19.9 μM | — | 11.5 μM | 67.2 μM | 94.1 μM |
| Prostate | | | | | |
| PC-3 | 24.9 μM | 21.8 μM | 17.9 μM | 23.5 μM | 49.8 μM |
| DU-145 | >100 μM | 38.8 μM | 17.0 μM | >100 μM | >100 μM |
| Breast | | | | | |
| MCF7 | 17.1 μM | 18.6 μM | 16.3 μM | 21.0 μM | 88.8 μM |
| NCI/ADR- | >100 μM | 21.4 μM | 27.6 μM | 41.1 μM | >100 μM |
| RES | | | | | |
| MDA-MB- | >100 μM | 17.8 μM | 18.1 μM | >100 μM | >100 μM |
| 231/ATCC | | | | | |
| HS 578T | >100 μM | 16.4 μM | 19.1 μM | >100 μM | 48.3 μM |
| MDA-MB- | >100 μM | 18.2 μM | 16.9 μM | 60.1 μM | 83.2 μM |
| 435 | | | | | |
| BT-549 | 80.1 μM | 14.6 μM | 15.5 μM | 52.9 μM | 67.5 μM |
| T-47D | 5.05 μM | 16.2 μM | 14.7 μM | 13.1 μM | 33.5 μM |

TABLE 3
Cytostatic and Cytotoxic Effects Of Compounds Q012132 and
Q012135T On Human Cancer Cell-Lines

| | | Q012132 | | | Q012135T | |
|---|---|---|---|---|---|---|
| | GI50 | TGI | LC50 | GI50 | TGI | LC50 |
| Leukemia | | | | | | |
| CCRF-CEM | — | — | — | 14.1 μM | 48.4 μM | 14.1 μM |
| HL-60(TB) | 14.8 μM | 32.8 μM | 72.6 μM | 13.1 μM | 33.7 μM | 13.1 μM |
| K-562 | 5.21 μM | 19.6 μM | 73.5 μM | 3.4 μM | 14.1 μM | 3.4 μM |
| MOLT-4 | 13.0 μM | 42.4 μM | >100 μM | 10.1 μM | 31.3 μM | 10.1 μM |
| RPMI-8226 | — | — | — | 7.14 μM | 24.5 μM | 7.14 μM |
| SR | 11.0 μM | 30.5 μM | 84.4 μM | 4.18 μM | 20.6 μM | 4.18 μM |
| Non-Small Cell | | | | | | |
| Lung | | | | | | |
| A549/ATCC | 31.2 μM | >100 μM | >100 μM | 36.9 μM | >100 μM | >100 μM |
| EKVX | 91.2 μM | >100 μM | >100 μM | 21.3 μM | 62.1 μM | >100 μM |
| HOP-62 | — | — | — | 20.6 μM | 43.9 μM | 93.8 μM |
| HOP-92 | 10.7 μM | 26.6 μM | 66.3 μM | 12.8 μM | 31.8 μM | 79.1 μM |
| NCI-H226 | 65.5 μM | >100 μM | >100 μM | 19.8 μM | 42.6 μM | 91.5 μM |
| NCI-H23 | 29.6 μM | >100 μM | >100 μM | 34.2 μM | >100 μM | >100 μM |
| NCI-H322M | 37.8 μM | >100 μM | >100 μM | 33.8 μM | >100 μM | >100 μM |
| NCI-H460 | 21.3 μM | 41.7 μM | 81.7 μM | 20.2 μM | 37.7 μM | 70.4 μM |
| NCI-H522 | | | | 15.5 μM | 35.6 μM | 81.7 μM |
| Colon | | | | | | |
| COLO 205 | 16.8 μM | 34.8 μM | 72.1 μM | 17.0 μM | 36.3 μM | 77.6 μM |
| HCC-2998 | 14.5 μM | 28.9 μM | 57.7 μM | 15.3 μM | 31.6 μM | 65.1 μM |
| HCT-116 | 10.8 μM | 22.6 μM | 47.6 μM | 18.6 μM | >100 μM | >100 μM |
| HCT-15 | 16.8 μM | 36.9 μM | 80.8 μM | 26.9 μM | >100 μM | >100 μM |
| HT29 | 3.11 μM | 16.7 μM | 63.0 μM | 17.1 μM | 31.8 μM | 59.0 μM |
| KM12 | 31.6 μM | >100 μM | >100 μM | 21.0 μM | 39.2 μM | 73.3 μM |
| SW-620 | 16.1 μM | 30.8 μM | 59.1 μM | 18.5 μM | 35.5 μM | 68.0 μM |
| CNS | | | | | | |
| SF-268 | 18.5 μM | 44.1 μM | >100 μM | 19.1 μM | 36.9 μM | 71.1 μM |
| SF-295 | 19.3 μM | 41.4 μM | 89.0 μM | 19.6 μM | 38.0 μM | 73.5 μM |
| SF-539 | 14.6 μM | 28.2 μM | 54.6 μM | 16.0 μM | 30.2 μM | 57.2 μM |
| SNB-19 | 41.8 μM | >100 μM | >100 μM | 33.1 μM | >100 μM | >100 μM |
| SNB-75 | 22.3 μM | 49.8 μM | >100 μM | 17.6 μM | 32.2 μM | 59.2 μM |
| U251 | 15.1 μM | 29.1 μM | 55.8 μM | 15.8 μM | 32.2 μM | 65.7 μM |
| Melanoma | | | | | | |
| LOX IMVI | 14.1 μM | 27.9 μM | 55.1 μM | 15.8 μM | 31.7 μM | 63.4 μM |
| MALME-3M | 15.7 μM | 32.0 μM | 65.5 μM | 20.1 μM | 38.6 μM | 74.1 μM |
| M14 | 5.8 μM | 17.5 μM | 41.9 μM | 17.2 μM | 33.4 μM | 64.8 μM |
| SK-MEL-2 | | | | 17.2 μM | 36.9 μM | 79.5 μM |
| SK-MEL-28 | 16.8 μM | 35.6 μM | 75.5 μM | 15.1 μM | 29.5 μM | 57.4 μM |
| SK-MEL-5 | 13.1 μM | 26.0 μM | 51.5 μM | 12.9 μM | 25.8 μM | 51.8 μM |
| UACC-257 | 18.4 μM | 45.7 μM | >100 μM | — | — | — |
| UACC-62 | 17.2 μM | 33.3 μM | 64.3 μM | 20.1 μM | 38.2 μM | 72.4 μM |
| Ovarian | | | | | | |
| IGR-OV1 | — | — | — | 14.7 μM | 43.4 μM | >100 μM |
| OVCAR-3 | 33.1 μM | >100 μM | >100 μM | 14.1 μM | 29.4 μM | 61.2 μM |
| OVCAR-4 | 47.8 μM | >100 μM | >100 μM | 18.6 μM | 42.7 μM | 98.0 μM |
| OVCAR-5 | 29.3 μM | >100 μM | >100 μM | 23.4 μM | 46.3 μM | 91.7 μM |
| OVCAR-8 | 22.3 μM | 61.8 μM | >100 μM | 22.1 μM | 49.1 μM | >100 μM |
| SK-OV-3 | 29.4 μM | 98.8 μM | >100 μM | 24.9 μM | 56.7 μM | >100 μM |
| Renal | | | | | | |
| 786-0 | 14.9 μM | 28.2 μM | 53.1 μM | 16.2 μM | 31.0 μM | 59.2 μM |
| A498 | 17.8 μM | 36.3 μM | 74.1 μM | 25.8 μM | 51.4 μM | >100 μM |
| ACHN | 21.0 μM | 48.5 μM | >100 μM | 29.9 μM | >100 μM | >100 μM |
| CAKI-1 | 22.8 μM | 51.7 μM | >100 μM | 17.2 μM | 44.0 μM | >100 μM |
| RXF 393 | 13.6 μM | 30.2 μM | 67.2 μM | 18.8 μM | 39.9 μM | 84.5 μM |
| SN12C | 340 μM | >100 μM | >100 μM | 21.7 μM | 55.7 μM | >100 μM |
| TK-10 | 19.5 μM | 41.0 μM | 86.5 μM | 20.6 μM | 46.3 μM | >100 μM |
| UO-31 | — | — | — | 11.5 μM | 26.6 μM | 61.6 μM |
| Prostate | | | | | | |
| PC-3 | 21.8 μM | 81.1 μM | >100 μM | 17.9 μM | 45.4 μM | >100 μM |
| DU-145 | 38.8 μM | >100 μM | >100 μM | 17.0 μM | 36.5 μM | 78.4 μM |
| Breast | | | | | | |
| MCF7 | 18.6 μM | 36.9 μM | 73.0 μM | 16.3 μM | 33.7 μM | 69.9 μM |
| NCI/ADR-RES | 21.4 μM | 79.2 μM | >100 μM | 27.6 μM | 96.9 μM | >100 μM |
| MDA-MB-231/ATCC | 17.8 μM | 41.0 μM | 94.3 μM | 18.1 μM | 38.1 μM | 80.5 μM |
| HS 578T | 16.4 μM | 38.2 μM | 88.6 μM | 19.1 μM | 41.7 μM | 90.9 μM |
| MDA-MB-435 | 18.2 μM | 36.4 μM | 73.1 μM | 16.9 μM | 38.4 μM | 87.1 μM |
| BT-549 | 14.6 μM | 28.9 μM | 57.1 μM | 15.5 μM | 30.0 μM | 58.0 μM |
| T-47D | 16.2 μM | 44.4 μM | >100 μM | 14.7 μM | 49.2 μM | >100 μM |

The precise dosage to be employed depends upon several factors including the host, whether in veterinary medicine or human medicine, the nature and severity of the condition being treated, the mode of administration and the particular active substance employed. The compounds may be administered by any conventional route, in particular enterally, preferably orally in the form of tablets or capsules.

Administered compounds can be in the free form or pharmaceutically acceptable salt form as appropriate, for use as a pharmaceutical, particularly for use in the prophylactic or curative treatment of atherosclerosis and sequalae (angina pectoris, myocardial infarction, arrhythmias, heart failure, kidney failure, stroke, peripheral arterial occlusion, and related disease states). These measures will slow the rate of progress of the disease state and assist the body in reversing the process direction in a natural manner.

Any suitable carrier known to the art can be used to prepare the pharmaceutical compositions. In such a composition, the carrier may be a solid, liquid or mixture of a solid and a liquid. Solid compositions include powders, tablets and capsules. A solid carrier can be one or more substances which may also act as a flavoring agent, lubricant, solubilizer, suspending agent, binder, or tablet disintegrant. In powders, the carrier is a finely divided solid, which is in admixture with the finely divided active ingredient. In tablets, the active ingredient is mixed with a carrier having the necessary binding properties in suitable proportions and compacted in the shape and size desired. Suitable solid carriers are magnesium carbonate, magnesium stearate, talc, sugar, lactose, pectin, dextrin, starch, gelatin, tragacanth, methyl cellulose, hydroxymethyl cellulose, sodium carboxymethyl cellulose, a low melting wax, cocoa butter, and the like. Encapsulating materials may also be employed with the compounds of this invention, and the term “composition” is intended to include the active ingredient in combination with an encapsulating material as a formulation, with or without other carriers. Cachets may also be used in the delivery of the anti-atherosclerotic medicament of this invention.

Sterile liquid compositions include solutions, suspension, emulsions, syrups and elixirs. The compounds of this invention may be dissolved or suspended in the pharmaceutically acceptable carrier, such as sterile water, sterile organic solvent or a mixture of both. Preferably the liquid carrier is one suitable for parental injection. Where the compounds are sufficiently soluble they can be dissolved directly in normal saline with or without the use of suitable organic solvents, such as propylene glycol or polyethylene glycol. If desired, dispersions of the finely divided compounds can be made-up in aqueous starch or sodium carboxymethyl cellulose solution, or in a suitable oil, such as arachis oil. Liquid pharmaceutical compositions, which are sterile solutions or suspensions, can be utilized by intramuscular, intraperitoneal or subcutaneous injection. In many instances a liquid composition form may be used instead of the preferred solid oral method of administration.

It is preferred to prepare unit dosage forms of the compounds for standard administration regimes. In this way, the composition can be subdivided readily into smaller doses at the physician's direction. For example, unit dosages may be made up in packeted powders, vials or ampoules and preferably in capsule or tablet form. The active compound present in these unit dosage forms of the composition may be present in an amount of from about one gram to about fifteen grams or more, for single or multiple daily administration, according to the particular need of the patient. The daily dose of active compound will vary depending upon the route of administration, the size, age and sex of the patient, the severity of the disease state, and the response to the therapy as traced by blood analysis and the patient's recovery rate. By initiating the treatment regimen with a minimal daily dose of about one gram, the blood levels of PAI-1 and the patient's symptomatic relief analysis may be used to determine whether a larger dose is indicated. Based upon the data presented below, the projected daily dose for both human and veterinary use will be from about 25 to about 200 milligrams/kilogram per day, and more usually, from about 50 to 100 milligrams/kilogram per day.

### REFERENCES

1. Chorostowska-Wynimko, J., E. Skrzypczak-Jankun, and J. Jankun, Plasminogen activator inhibitor type-1: its structure, biological activity and role in tumorigenesis (Review). Int J Mol Med, 2004. 13(6): p. 759-66.
2. Lijnen, H. R., Pleiotropic functions of plasminogen activator inhibitor-1. J Thromb Haemost, 2005. 3(1): p. 35-45.
3. Hu, B., et al., Synthesis and SAR of 2-carboxylic acid indoles as inhibitors of plasminogen activator inhibitor-1. Bioorg Med Chem Lett, 2005. 15(15): p. 3514-8.
4. Stefansson, S., et al., Plasminogen activator inhibitor-1 in tumor growth, angiogenesis and vascular remodeling. Curr Pharm Des, 2003. 9(19): p. 1545-64.
5. Mayasundari, A., et al., The solution structure of the N-terminal domain of human vitronectin: proximal sites that regulate fibrinolysis and cell migration. J Biol Chem, 2004. 279(28): p. 29359-66.
6. Reuning, U., et al., Molecular and functional interdependence of the urokinase-type plasminogen activator system with integrins. Biol Chem, 2003. 384(8): p. 1119-31.
7. Takahashi, T., et al., Plasminogen activator inhibitor type 1 promotes fibrosarcoma cell migration by modifying cellular attachment to vitronectin via alpha(v)beta(5) integrin. Semin Thromb Hemost, 2005. 31(3): p. 356-63.
8. Czekay, R. P., et al., Plasminogen activator inhibitor-1 detaches cells from extracellular matrices by inactivating integrins. J Cell Biol, 2003. 160(5): p. 781-91.
9. Wei, Y., et al., Regulation of alpha5beta1 integrin conformation and function by urokinase receptor binding. J Cell Biol, 2005. 168(3): p. 501-11.
10. Zhang, X., et al., Functional plasminogen activator inhibitor-1 gene variants and breast cancer survival. Clin Cancer Res, 2006. 12(20 Pt 1): p. 6037-42.
11. Lindberg, P., A. Larsson, and B. S, Nielsen, Expression of plasminogen activator inhibitor-1, urokinase receptor and laminin gamma-2 chain is an early coordinated event in incipient oral squamous cell carcinoma. Int J Cancer, 2006. 118(12): p. 2948-56.
12. Wu, Q. and Z. Zhao, Inhibition of PAI-1: a new anti-thrombotic approach. Curr Drug Targets Cardiovasc Haematol Disord, 2002. 2(1): p. 27-42.
13. Liang, A., et al., Characterization of a small molecule PAI-1 inhibitor, ZK4044. Thromb Res, 2005. 115(4): p. 341-50.
14. Bajou, K., et al., Host-derived plasminogen activator inhibitor-1 (PAI-1) concentration is critical for in vivo tumoral angiogenesis and growth. Oncogene, 2004. 23(41): p. 6986-90.
15. Stefansson, S., et al., Novel approaches to thrombolysis based on modulation of endogenous fibrinolysis. Coron Artery Dis, 1998. 9(2-3): p. 99-104.
16. a) Gils, A. and P. J. Declerck, The structural basis for the pathophysiological relevance of PAI-I in cardiovascular diseases and the development of potential PAI-I inhibitors. Thromb Haemost, 2004. 91(3): p. 425-37. b) Hoekstra, T., et al., Plasminogen activator inhibitor-type 1: its plasma determinants and relation with cardiovascular risk. Thromb Haemost, 2004. 91(5): p. 861-72.
17. Steinmetzer, T., Synthetic urokinase inhibitors as potential antitumor drugs. IDrugs, 2003. 6(2): p. 138-46.
18. Trost, S., R. Pratley, and B. Sobel, Impaired fibrinolysis and risk for cardiovascular disease in the metabolic syndrome and type 2 diabetes. Curr Diab Rep, 2006. 6(1): p. 47-54.
19. Mertens, I., et al., Among inflammation and coagulation markers, PAI-1 is a true component of the metabolic syndrome. Int J Obes (Lond), 2006. 30(8): p. 1308-14.
20. Griffiths, S. L. and D. J. Grainger, Proposal of a novel diabetogenic mechanism involving the serpin PAI-1. Bioessays, 2006. 28(6): p. 629-41.
21. Czekay, R. P. and D. J. Loskutoff, Unexpected role of plasminogen activator inhibitor 1 in cell adhesion and detachment. Exp Biol Med (Maywood), 2004. 229(11): p. 1090-6.
22. Weisberg, A. D., et al., Pharmacological inhibition and genetic deficiency of plasminogen activator inhibitor-1 attenuates angiotensin II/salt-induced aortic remodeling. Arterioscler Thromb Vasc Biol, 2005. 25(2): p. 365-71.
23. Crandall, D. L., et al., Characterization and comparative evaluation of a structurally unique PAI-1 inhibitor exhibiting oral in-vivo efficacy. J Thromb Haemost, 2004. 2(8): p. 1422-8.
24. Elokdah, H., et al., Tiplaxtinin, a novel, orally efficacious inhibitor of plasminogen activator inhibitor-1: design, synthesis, and preclinical characterization. J Med Chem, 2004. 47(14): p. 3491-4.
25. Hennan, J. K., et al., Evaluation of PAI-039 [{1-benzyl-5-[4-(trifluoro-methoxy)phenyl]-1H-indol-3-yl}(oxo)acetic acid], a novel plasminogen activator inhibitor-1 inhibitor, in a canine model of coronary artery thrombosis. J Pharmacol Exp Ther, 2005. 314(2): p. 710-6.
26. Jankun, J., et al., Plasminogen activator inhibitor-1 is locked in active conformation and polymerizes upon binding ligands neutralizing its activity. Int J Mol Med, 2006. 17(3): p. 437-47.
27. Boyd, M. R., and K. D. Paull, Some practical considerations and applications of the National Cancer Institute in-vitro anticancer drug discovery screen. Drug Dev. Res., 1995. 34: p. 91-109.
28. Monks, A., et al., Feasibility of a high-flux anticancer drug screen using a diverse panel of cultured human tumor cell-lines. J. National Cancer Inst., 1991. 83(11):p. 757-66.

1-29. (canceled)

30. A compound of formula I or I′: 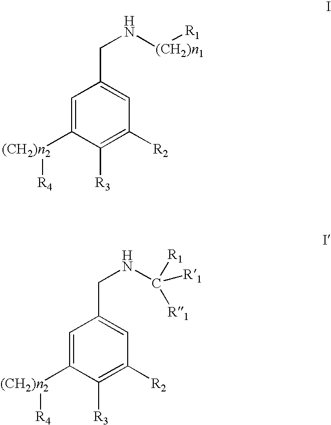
wherein:
R₁, R′₁, and R″₁, are each independently selected from H, linear, branched, saturated, unsaturated, cyclic, bicyclic, fused, substituted or unsubstituted alkyl, and an aryl, arylalkyl or heterocyclic group which is optionally substituted;
R₂ and R₃ are each independently selected from H, OH, SH, alkoxy, alkylthio, aryloxy and arylthio;
R₄ is a linear, branched, saturated, unsaturated, cyclic, bicyclic, fused, substituted, or unsubstituted alkyl, an aryl or arylalkyl group optionally containing at least one nitrogen atom and/or being aromatic and/or being substituted or unsubstituted, or a 4-substituted or unsubstituted piperazin-1-yl group;
n₁, and n₂ are each independently selected from 0 to 6; and
the stereochemistry of the carbon atom bearing substituents R₁, R′₁ and R″₁ is S or R,
with the proviso for compound I that R₄ is not NH₂ when n₁, is 0, n₂ is 1, R is H, R₂ is OCH₃ and R₃ is OH,
or a pharmaceutically acceptable salt thereof.

31. The compound as defined in claim 30, wherein said compound is formula Ib or I′b: 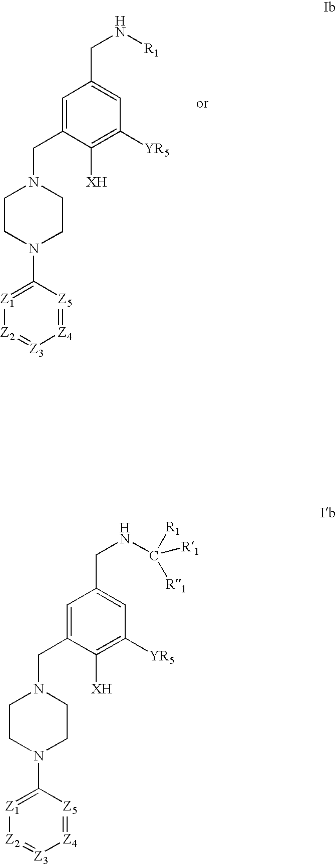
wherein:
R₁, R′₁, and R″₁, are as defined in claim 30;
R₅ is alkyl or aryl;
X and Y are each independently selected from O and S;
Z₁ to Z₅ are each independently selected from C and N;
or a pharmaceutically acceptable salt thereof.

32. The compound as defined in claim 31, which is A, B, C or D 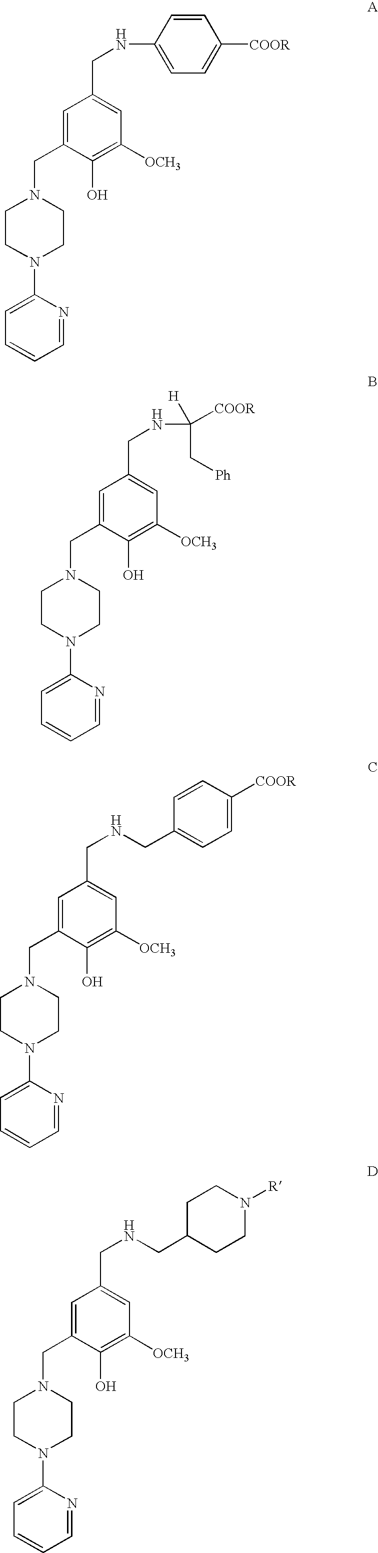
wherein R and R′ are each independently selected from H and a linear, branched, saturated or unsaturated alkyl, and optionally R′ is Boc or C(═NH)NH₂; and the stereochemistry of the asymmetric carbon is S or R, or a pharmaceutically acceptable salt thereof.

33. The compound as defined in claim 32, wherein R is H, Et or t-Bu and R′ is H, Boc or C(═NH)NH₂.

34. The compound as defined in claim 31, which is Q012110, Q012112, Q012119T, Q012131, Q012132, Q012135T, Q012136, Q012143, Q012145T, Q012146, Q012147T or Q012148: 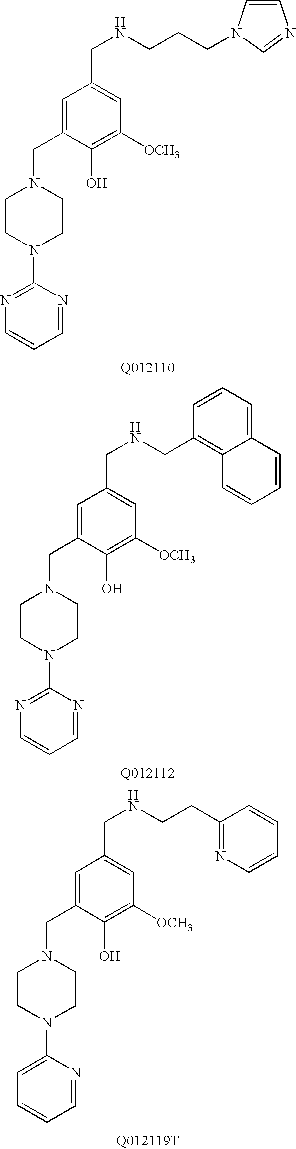 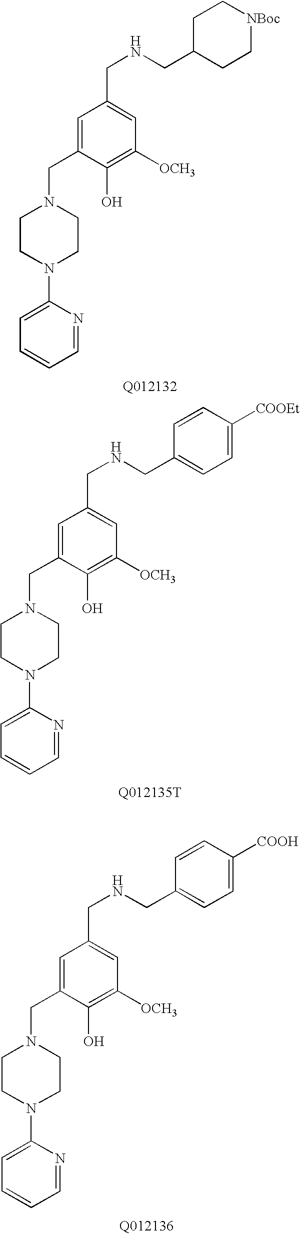 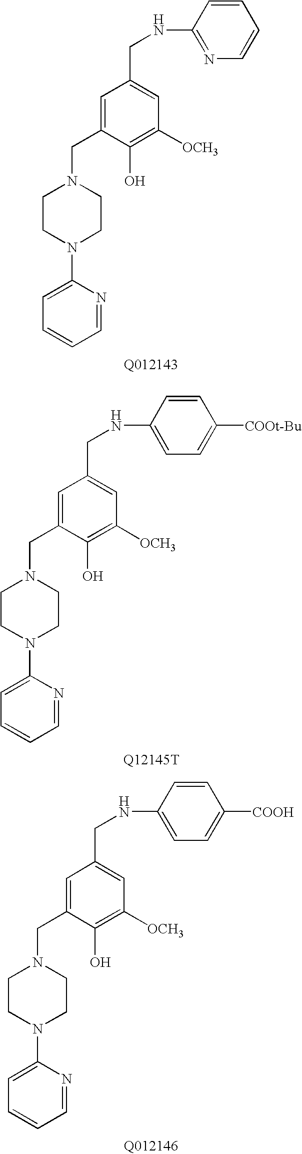 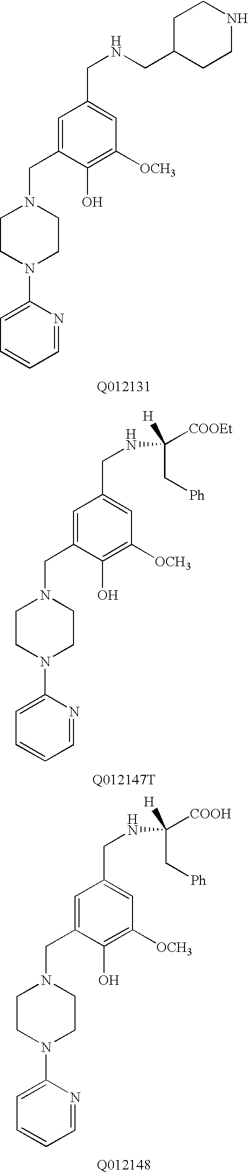
or a stereoisomer of Q012147T or Q012148, or a pharmaceutically acceptable salt thereof.

35. A method of treating or preventing a disease in a human patient, in which the disease involves the production and/or action of PAI-1, by administering a therapeutically effective amount of the compound as defined in claim 30.

36. A method as defined in claim 35, wherein said disease is selected from one or more of: noninsulin dependent diabetes mellitus and cardiovascular disease caused by such conditions; prevention of thrombotic events associated with coronary artery and cerebrovascular disease; inhibiting the disease process involving the thrombotic and prothrombotic states which include atherosclerotic plaques, venous and arterial thrombosis, myocardial ischemia, atrial fibrillation, deep vein thrombosis, blood clotting disorders, pulmonary fibrosis, cerebral thrombosis, thromboembolic complications of surgery including joint replacement, and peripheral arterial occlusion; stroke associated with or resulting from atrial fibrillation; diseases associated with extracellular matrix accumulation, including, but not limited to, renal fibrosis, chronic obstructive pulmonary disease, polycystic ovary syndrome, restenosis, renovascular disease and organ trans-plant rejection; malignancies and diseases associated with neoangiogenesis including diabetic retinopathy; cancer, including, but not limited to, breast, ovary, colon, central nervous system, kidney and prostate cancers, and as imaging agents for the identification of metastatic cancers; myelofibrosis with myeloid metaplasia by regulating stromal cell hyperplasia and increases in extracellular matrix proteins; diabetic nephropathy and renal dialysis associated with nephropathy; septicemia, obesity, insulin resistance, proliferative diseases such as psoriasis, improving coagulation homeostasis, cerebrovascular disease, micro-vascular disease, hypertension, dementia, osteoporosis, asthma, and as a hormone replacement agent, treating, preventing or reversing progression of atherosclerosis, Alzheimer's disease, osteoporosis, osteopenia; reducing inflammatory markers, reducing C-reactive protein, or preventing or treating low grade vascular inflammation, stroke, dementia, coronary heart disease, primary and secondary prevention of myocardial infarction, stable and unstable angina, peripheral vascular disease, peripheral arterial disease, acute vascular syndromes, microvascular disease such as nephropathy, neuropathy, retinopathy and nephrotic syndrome, hypertension, Type 1 and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improving coagulation homeostasis, and/or improving endothelial function, and all forms of cerebrovascular diseases; septicemia, obesity, insulin resistance, psoriasis and related conditions, cerebrovascular diseases, arthritis, heart failure, angina and other cardiac conditions, malignant and premalignant lesions, for topical wound healing, inflammatory diseases, septic shock and vascular damage associated with infections.

37. The method as defined in claim 35, further comprising administering one or more of a prothrombolytic, fibrinolytic or anti-coagulant agent, or a protease inhibitor-containing highly active anti-retroviral therapy, for treatment or prophylaxis of fibrinolytic impairment and hypercoaguability of HIV-1 infected patients.

38. The method as defined in claim 37, wherein the disease is cancer.

39. The method as defined in claim 38, wherein the cancer is leukemia, non-small cell lung cancer, colon cancer, central nervous system cancers, melanoma, kidney cancer, ovarian cancer, renal cancer, prostate cancer or breast cancer.

40. The method as defined in claim 39, wherein the compound is selected from Q012132, Q012135T, Q012145T, and Q012147T, or a pharmaceutically acceptable salt thereof: 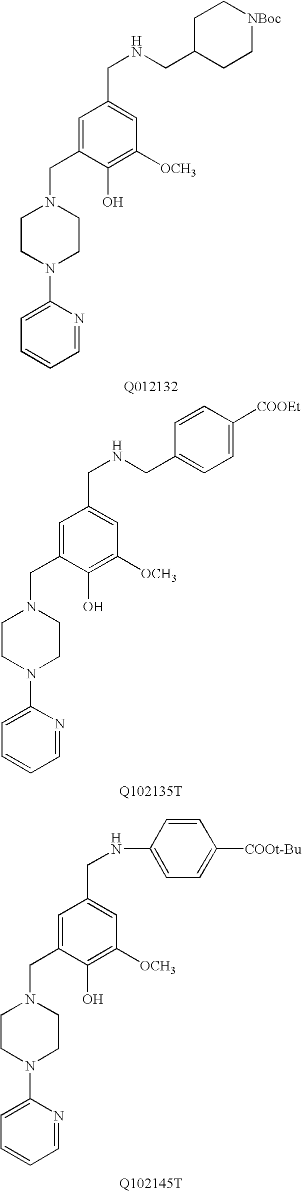 [chemical structure]
